(19) United States
(12) Patent Application Publication
(10) Pub. No.: US 2005/0124645 A1
(43) Pub. Date: Jun. 9, 2005

(54) Methods and compositions for increasing CD4lymphocyte immune responsiveness

(76) Inventor: Terri H. Finkel, Wynnewood, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/926,749

(22) Filed: Aug. 26, 2004

Related U.S. Application Data
(63) Continuation-in-part of application No. 10/673,882, filed on Sep. 29, 2003.
Application No. 10/673,882 is a continuation-in-part of application No. 10/313,923, filed on Dec. 5, 2002.
Application No. 10/313,923 is a continuation of application No. 09/294,949, filed on Apr. 20, 1999, now abandoned.
(60) Provisional application No. 60/082,453, filed on Apr. 20, 1998.

(51) Int. Cl. A61K 31/4745

(52) U.S. Cl. 514/291

(57) ABSTRACT

Compositions and methods are provided for the treatment of infections by microbes associated with a latent infection.

This application is a continuation-in-part of U.S. application Ser. No. 10/673,882, filed Sep. 29, 2003, which is a continuation-in-part of U.S. application Ser. No. 10/313,923, filed Dec. 5, 2002, which is a continuation of U.S. application Ser. No. 09/294,949, filed Apr. 20, 1999, which claims priority to U.S. Provisional Application No. 60/082,453, filed Apr. 20, 1998. The entire disclosure of the above-identified applications is incorporated by reference herein.

Pursuant to 35 U.S.C. Section 202(c), it is acknowledged that the United States Government has certain rights in the invention described herein, which was made in part with funds from the National Institutes of Health Grant Nos. AI35513 and AI40003.

### FIELD OF THE INVENTION

The present invention generally relates to compositions and methods to restore immune responsiveness to CD4⁺ T lymphocytes in HIV infected patients and for modulation of HIV latency.

### BACKGROUND OF THE INVENTION

Several publications and patent documents are cited throughout the specification in order to describe the state of the art to which this invention pertains. Each citation is incorporated herein as though set forth in full.

Nearly twenty years have passed since the human immunodeficiency virus (HIV) was first identified as the causative agent of acquired immunodeficiency syndrome (AIDS). The human toll of this virus is tragic—almost 20 million people have died of AIDS, over 30 million are currently living with HIV, and 16,000 new infections occur daily. Still, there is neither a cure nor a proven therapy or vaccine for the treatment of HIV/AIDS.

Human immunodeficiency virus type-1 (HIV-1) infection is characterized by a host-virus relationship in which the virus utilizes the host cell's macromolecular machinery and energy supplies to produce progeny virus (Fauci, A. S. (1996) Nature, 384:529-534). Inevitably, after initial infection by HIV-1, the virus alters the host cell's physiological state, leading to disruption of immune responses, cell growth arrest, and cell death (McMichael, A. and Phillips, R. E. (1997) Ann. Rev. Immunol., 15:271-296). Specific viral and host cellular proteins are known to play crucial roles in this process (Panatleo, G. and Fauci, A. S. (1996) Ann. Rev. Microb., 50:825-854). For example, the HIV-1 accessory proteins and host cell chemokine co-receptors, CCR5 and CXCR4, are essential for HIV-1 infection (Deng, H., et al. (1996) Nature, 381:661-666; Liu, R., et al. (1996) Cell, 86:367-377; Murdoch, C. (2000) Immunol. Rev. 177:175-184), and host cell target genes such as Ets-1, CDK4, NFAT1 and NFAT2, induce enhanced HIV-1 gene expression in vitro (Posada, R., et al. (2000) AIDS Res. and Hum. Retrovirus, 18:1981-1989; Nekhai, S., et al. (2000) Virology 266:246-256; Cron, R. Q., et al. (2000) Clin. Immunol. 94:179-191; Kinoshita, S., et al. (1998) Cell, 95:595-604; Kinoshita, S., et al. (1997) Immunity 6:235-244).

HIV-1 preferentially infects a class of immune cells called CD4⁺ T cells or helper T cells, which are essential to the function of the immune system. Following primary HIV-1 infection, the virus replicates in local lymph nodes and then disseminates in a massive viremia. Although HIV-1 elicits strong immune responses in most infected individuals, the virus almost invariably escapes immune containment (Fauci, A. S. (1996) Nature, 384:529-534; McMichael, A. and Phillips, R. E. (1997) Ann. Rev. Immunol., 15:271-296). During early HIV infection and in asymptomatic individuals, CD4⁺ T cells fail to proliferate to antigenic or mitogenic stimulation, and immunodeficiency is evident even before the progressive decline in CD4⁺ T cells which leads to AIDS and ultimately death (Shearer et al. (1986) J. Immunol., 137:2514; and Lane et al. (1985) N. Engl. J. Med., 313:79). Prior to the present invention, the mechanism(s) of this inhibition of CD4⁺ cells were not fully understood. Notably, ligation of CD4 by gp120 was found, however, to inhibit TCR/CD3-induced interleukin-2 receptor (IL-2R) expression, IL-2 production, and proliferation (Oyalzu et al. (1990) Proc. Natl. Acad. Sci. USA, 87:2379; Banda et al. (1996) Apoptosis, 1:49; and Liegler et al. (1994) J. Acquir. Immune. Defic. Syndr. 7:340).

Current therapies against HIV-1 infection are specific for targeting the virus. However, these therapies are not able to induce sustained suppression or cure of HIV because of HIV's ability to develop resistance to the treatment. Even when the amount of virus in the blood falls below the current limits of detection, HIV continues to reproduce at very low levels or alternatively, resides in a “reservoir” of latently infected T cells. Indeed, during latent HIV infection of CD4⁺ T lymphocytes, little or no viral protein is produced thereby preventing the immune system from detecting the presence of an infection in these cells. Latently infected CD4⁺ T cells can account for as much as 10% of the total infected CD4⁺ T lymphocytes in an individual.

One treatment for HIV-1 infection is a cocktail of anti-viral drugs known as Highly Active Anti-Retroviral Therapy (or HAART) which includes two reverse transcriptase inhibitors and a protease inhibitor. HAART reduces the viral load in many patients to levels below the current limits of detection, but the rapid mutation rate of this virus limits the efficacy of this therapy (Perrin, L. and Telenti, A. (1998) Science 280:1871-1873). In addition, HAART is ineffective in some patients with HIV-1 infection and many more cannot tolerate its debilitating side effects.

Therapies for HIV-1 infection in the experimental stages of testing include the development of vaccines against HIV-1. Vaccines based on engineered gp120-CD4-CCR5 fusion proteins have been shown to elicit antibodies capable of neutralizing HIV-1 infectivity (LaCasse, R. A., et al. (1999) Science, 283:357-362). However, evidence of in vivo efficacy is not yet available and most researchers believe that a highly promising ideal vaccine candidate is not yet at hand (Nabel, G. J. (2001) Nature, 410:1002-1007).

Inasmuch as a functional and healthy immune system is better able to control HIV viral load, therapeutic strategies aimed to enhance the ability of the immune system have been employed to combat HIV infection. For example, IL-2 (interleukin 2) has been employed to enhance immune function, but has been shown to have systemic toxic side effects, thereby limiting the agent's usefulness (DePaoli, et al. (1997) J. Clin. Invest., 100:2737).

Given the continuing impact of the HIV epidemic around the world and the lack of a proven therapy which provides sustained protection against HIV infection and AIDS, there remains a critical need for HIV research to identify new ways to prevent and treat this deadly disease, including means by which to restore the immune response.

### SUMMARY OF THE INVENTION

The present invention generally relates to a method and composition to increase and/or restore immune responsiveness in CD4⁺ T lymphocytes that display a reduction or loss of immune responsiveness after CD4 is ligated by human immunodeficiency virus (HIV) gp120. The present inventors have discovered that aberrant regulation of the Janus family kinase, JAK3, signaling pathway, as a result of CD4 ligation on a T cell by HIV envelope glycoprotein prior to activation of the T cell (i.e., CD4 ligation of a resting or naive T cell), results in a loss of T cell responsiveness (i.e., defective CD4⁺ T cell function). Moreover, this defect in T cell function can ultimately contribute to the loss and/or inhibition of development of CD4⁺ T cells in HIV-infected individuals. More specifically, the present inventors have demonstrated that CD4 ligation prior to T cell receptor (TCR)—mediated T cell activation (either artificially or as a result of HIV infection) markedly inhibits JAK3 and STAT5 expression and activation, which correlates with characteristics of a decrease in T cell responsiveness, including a reduced proliferative response, reduced IL-2 receptor (IL-2R) expression and/or reduced IL-2 secretion by the T cell. Furthermore, the present inventors have shown that engagement of γ_{c}-related cytokine receptors in these T cells increases anti-TCR-induced IL-2 receptor (IL-2R) expression, T cell proliferation, and IL-2 secretion, and that this rescue correlates with JAK3 and STAT5 activation in the cells.

One embodiment of the present invention relates to a method to increase CD4⁺ T lymphocyte immune responsiveness in a patient infected with human immunodeficiency virus (HIV). The method includes increasing JAK3 and/or STAT5 action in CD4⁺ T lymphocytes of the patient, wherein the increase in JAK3 and/or STAT5 action is sufficient to increase immune responsiveness in the CD4⁺ T lymphocytes. In one aspect of the method, the CD4⁺ T lymphocytes express CD4 that has been ligated by gp120 on the human immunodeficiency virus. Such a CD4⁺ T lymphocyte can be infected-or not infected by the human immunodeficiency virus. The method is useful for increasing JAK3 and/or STAT5 action in a CD4⁺ T lymphocyte that is latently infected by the human immunodeficiency virus, as well as in a CD4⁺ T lymphocyte productively infected by the human immunodeficiency virus.

In one embodiment, the present method is used in a patient having early onset HIV-infection. Such a patient can be characterized as having a CD4⁺ T cell count of at least about 100 cells/mm³ when the method is employed and/or an HIV viral load of less than about 400 copies/ml when the method is employed.

In another embodiment, the method is employed in conjunction with administration to the patient of one or more anti-retroviral therapeutic compounds. Such compounds include, but are not limited to, AZT, ddI, ddC, d4T, 3TC and/or protease inhibitors.

In one embodiment of the present method, the method includes the step of administering to the CD4⁺ T lymphocytes a composition comprising one or more compounds that increase the action of JAK3 and/or STAT5 in the CD4⁺ T lymphocytes. Such compounds can include, but are not limited to: (1) one or more compounds that selectively bind to and stimulate a receptor comprising a γ_{c} chain on the surface of the CD4⁺ T lymphocytes; (2) a cytokine selected from the group of interleukin-2 (IL-2), IL-4, IL-7, IL-9, IL-13 and/or IL-15, with IL-7, IL-9, IL-13 and/or IL-15 being preferred in one embodiment; (3) an antibody that selectively binds to and stimulates a receptor comprising a γ_{c} chain on the surface of the CD4⁺ T lymphocytes; (4) a compound that selectively increases JAK3 and/or STAT5 expression in the CD4⁺ T lymphocytes by associating with a transcription control sequence of a gene encoding the JAK3 and/or STAT5 such that JAK3 and/or STAT5 transcription is increased in the CD4⁺ T lymphocyte, including, but not limited to a transcription factor that selectively binds to the transcription control sequence; (5) a recombinant nucleic acid molecule comprising an isolated nucleic acid sequence encoding a biologically active JAK3 and/or STAT5 protein operatively linked to a transcription control sequence, whereby the CD4⁺ T lymphocyte expresses the biologically active JAK3 and/or STAT5 protein; (6) a biologically active JAK3 and/or STAT5 protein operatively linked to an N-terminal protein transduction domain from HIV TAT; and/or (7) a compound that is a product of rational drug design.

In one embodiment of the method of the present invention, the composition is administered in a pharmaceutically acceptable delivery vehicle. Such a pharmaceutically acceptable delivery vehicle can include, but is not limited to water, phosphate buffered saline, Ringer's solution, dextrose solution, serum-containing solutions, Hank's solution, other aqueous physiologically balanced solutions, oils, esters and glycols. In another embodiment, such a pharmaceutically acceptable delivery vehicle is an N-terminal protein transduction domain from HIV TAT. In another embodiment, such a pharmaceutically acceptable delivery vehicle is selected from the group of lipid-containing delivery vehicles, retroviral vectors and recombinant viruses. In yet another embodiment, such a pharmaceutically acceptable delivery vehicle specifically targets CD4⁺ T lymphocytes in the patient, and in one aspect, the pharmaceutically acceptable delivery vehicle specifically targets HIV-infected CD4⁺ T lymphocytes in the patient. Such a pharmaceutically acceptable delivery vehicle can include, but is not limited to an antibody that selectively binds to gp120, an immunoliposome comprising an antibody that selectively binds to gp120, and a liposome expressing CD4 on its surface.

The step of administering a composition according to the present invention can be performed in vivo, such as by an intradermal, intravenous, subcutaneous, oral, aerosol, intramuscular and intraperitoneal route of administration, or ex vivo such as by transfection, electroporation, micioinjection, lipofection, adsorption, protoplast fusion, use of protein carrying agents, use of ion carrying agents, and use of detergents for cell permeabilization.

The method of the present invention is useful for increasing the ability of the CD4⁺ T lymphocyte to proliferate in response to T cell receptor-mediated activation of the lymphocyte, and/or for increasing cytokine production by the CD4⁺ T lymphocyte.

Another embodiment of the present invention relates to a method to identify a regulatory compound that increases immune responsiveness by increasing JAK3 and/or STAT5 action in a CD4⁺ T lymphocyte that expresses CD4 that has been ligated in the absence of T cell activation. Such method includes the steps of (a) contacting a resting CD4⁺ T lymphocyte with a CD4 ligating compound that selectively binds to CD4 on the CD4⁺ T lymphocyte; (b) contacting the CD4⁺ T lymphocyte, after step (a), with a stimulatory compound that stimulates T cell receptor-mediated activation of the CD4⁺ T lymphocyte; (c) contacting the CD4⁺ T lymphocyte with a putative regulatory compound and, (d) determining whether JAK3 and/or STAT5 action is increased in the CD4⁺ T lymphocyte. The performance of step (a) prior to step (b) results in a decrease in immune responsiveness of the CD4⁺ T lymphocyte as compared to a control CD4⁺ T lymphocyte that was not contacted with the CD4 ligating compound prior to step (b). An increase in JAK3 and/or STAT5 action in the CD4⁺ T lymphocyte, as compared to JAK3 and/or STAT5 action in a control CD4⁺ T lymphocyte that has not been contacted with the putative regulatory compound, indicates that the putative regulatory compound increases immune responsiveness in the CD4⁺ T lymphocyte.

According to this method, the CD4-ligating compound can include, but is not limited to, an antibody that binds to CD4, gp120, a fragment of gp120 sufficient to bind to CD4, a Class II major histocompatibility (MHC) molecule, a CD4 binding region of a Class II MHC molecule, a cell line that expresses recombinant Env protein and a human immunodeficiency virus (HIV). In one aspect of the invention, step (a) comprises infecting the CD4⁺ T lymphocyte with a human immunodeficiency virus. In another aspect, step (a) comprises isolating latently HIV-infected T cells from the patient.

The stimulatory compound can include, but is not limited to an antibody that binds to a T cell receptor, an antibody that binds to CD3, a soluble MHC-antigen complex, a membrane bound MHC-antigen complex, T cell mitogens and a superantigen.

In one aspect of the method to identify a regulatory compound, step (c) of contacting the CD4⁺ T lymphocyte with a putative regulatory compound is performed within less than about 24 hours of step (b). In another aspect, step (c) of contacting the CD4⁺ T lymphocyte with a putative regulatory compound is performed prior to step (b). In another aspect, step (c) of contacting comprises administering the putative regulatory compound by a technique selected from the group of transfection, electroporation, microinjection, cellular expression (e.g., naked nucleic acid molecules, recombinant virus, retrovirus expression vectors and adenovirus expression vectors), lipofection, adsorption, protoplast fusion, use of ion carrying agents, use of protein carrying agents and use of detergents for cell permeabilization.

In the present method of identifying a regulatory compound, step (d) of determining can include, but is not limited to a method selected from the group of determining JAK3 and/or STAT5 mRNA levels, determining JAK3 and/or STAT5 protein levels, determining phosphorylation of JAK3 and/or STAT5, determining JAK3 phosphorylation of a substrate, determining association of JAK3 and/or STAT5 with another protein, determining association of STAT5 with a nucleic acid determining JAK3 enzymatic activity. In one aspect, step (d) of determining comprises a measurement selected from the group of: immunoblots, phosphorylation assays, kinase assays, immunofluorescence microscopy, RNA assays, immunoprecipitation, and biological assays. In another aspect, step (d) of determining comprises measuring JAK3 phosphorylation of STAT5 in the CD4⁺ T lymphocyte.

Yet another-embodiment of the present invention relates to a composition for treating CD4⁺ T lymphocytes having decreased responsiveness in HIV-infected patients. Such a composition includes: (a) a cytokine selected from the group consisting of IL-7, IL-9, IL-13 and IL-15, in an amount sufficient to increase JAK3 and/or STAT5 action in a CD4⁺ T lymphocyte in an HIV-infected patient; and, (b) at least one anti-retroviral agent in an amount sufficient to decrease HIV replication in the CD4⁺ T lymphocyte.

Another embodiment of the present invention relates to a method to increase CD4⁺ T lymphocyte immune responsiveness in a patient having human immunodeficiency virus (HIV) infection. Such method includes the step of administering to the patient a composition comprising: (a) a compound that selectively binds to and stimulates a receptor comprising a γ_{c} chain on the surface of CD4⁺ T lymphocytes in the patient, wherein the compound is administered in an amount sufficient to increase JAK3 and/or STAT5 action in the CD4⁺ T lymphocytes; and, (b) a pharmaceutically acceptable delivery vehicle that specifically targets T lymphocytes in the patient. In one embodiment, the patient has a CD4⁺ T cell count of at least about 100 cells/mm³ and an HIV viral load of less than about 400 copies/ml when the method is employed.

Yet another embodiment of the present invention relates to a method to increase CD4⁺ T lymphocyte immune responsiveness in a patient having human immunodeficiency virus (HIV) infection. Such a method includes the step of administering to the patient a composition comprising: (a) a compound selected from the group of: (1) a cytokine selected from the group consisting of interleukin-7 (IL-7), IL-9, IL-13 and IL-15; (2) a compound that increases the expression of JAK3 and/or STAT5 in the CD4⁺ T lymphocytes by associating with a transcription control sequence of a gene encoding JAK3 and/or STAT5 such that JAK3 and/or STAT5 transcription is increased; (3) a biologically active JAK3 and/or STAT5 protein, operatively linked to an N-terminal protein transduction domain from HIV TAT; and/or (4) a recombinant nucleic acid molecule comprising an isolated nucleic acid sequence encoding a biologically active JAK3 and/or STAT5 protein operatively linked to a transcription control sequence; and, (b) one or more anti-retroviral therapeutic compounds. The compound of part (a) is administered in an amount sufficient to increase JAK3 and/or STAT5 action in the CD4⁺ T lymphocytes.

Yet another embodiment of the present invention relates to a method to identify an HIV-infected patient as a suitable candidate for employment of a method to increase CD4⁺ T lymphocyte responsiveness. Such a method includes the steps of: (a) isolating a sample of T lymphocytes from an HIV infected patient; (b) stimulating the T lymphocytes with a stimulator that stimulates T cell receptor-mediated activation of the T lymphocytes in the presence and absence of a compound that binds to and activates a cytokine receptor having an γ_{c} chain; (c) measuring JAK3 and/or STAT5 action in the T lymphocytes of step (b); and, (d) identifying candidate patients in which the sample of T lymphocytes shows a measurable increase of at least about 10% in JAK3 and/or STAT5 action in the presence of the compound as compared to in the absence of the compound. In one aspect of the method, the method includes contacting the T lymphocytes in step (b) with a panel of compounds that bind to and activate a cytokine receptor having an γ_{c} chain. Such method further comprises step (e) of identifying a compound from the panel of compounds wherein the T lymphocytes show a larger increase in JAK3 and/or STAT5 action in the presence of the compound as compared to in the presence of the other compounds in the panel.

Yet another embodiment of the invention is a method to increase transcription of lentiviral genes in lentivirus-infected cells comprising contacting the cells with at least one compound which increases the JAK3 and/or STAT5 action within said cell, wherein the increase in JAK3 and/or STAT5 action is sufficient to increase transcription of lentiviral genes in the cells. In a certain embodiment of the invention, the lentivirus is HIV and the cells are CD4⁺ T lymphocytes. In another embodiment of the invention, the method reduces the amount of latently HIV infected CD4⁺ T lymphocytes in the patient. In yet another aspect of the invention, the method prevents the production of latently HIV infected CD4⁺ T lymphocytes in the patient. In yet another aspect of the invention, the compound of the method, such as Nef antisense nucleic acid or Nef siRNA, blocks the synthesis of Nef protein.

Additionally, the present invention generally relates to methods for the treatment of latent infections. In a particular embodiment of the invention, the microbe which latently infects the host is selected from the group consisting of herpes viruses, lentiviruses (e.g., human immunodeficiency virus-1 (HIV-1), HIV-2, feline immunodeficiency virus (FIV), simian immunodeficiency virus (SIV)), hepatitis B virus (HBV), Epstein-Barr virus (EBV), Mycobacterium tuberculosis (TB), Schistosoma flatworms (e.g., Schistosoma mansoni, S. intercalatum, S. haematobium, S. japonicum and S. mekongi), and Toxoplasma gondii. In a preferred embodiment of the invention, the microbe is HIV-1.

In accordance with another embodiment of the present invention, methods are provided for treating a patient that has been infected by a microbe which is associated with latent infections. The methods of the invention comprise the administration of at least one of the group consisting of: pentostam (stibogluconate sodium), rapamycin, and derivatives or analogs thereof. In a particular embodiment, the treatment results in the reduction, elimination, and/or prevention of the formation of the microbe latent reservoir. In a particular embodiment, the present method is used in a patient during an acute infection by the microbe. In yet another embodiment, the patient is undergoing a structured treatment interruption (STI).

In yet another embodiment of the invention, the latent reservoir of the microbe in the patient can be monitored by measuring at least one of the group consisting of: microbe RNA, microbe DNA, CD4 cell levels, infectious microbes by co-culture with other cells (e.g., PBMCs), p24 expression for HIV, and IFN-γ secretion by CD8 cells.

### BRIEF DESCRIPTION OF THE DRAWINGS

FIG. 1 is a bar graph illustrating that stimulation of purified CD4⁺ T lymphocytes through γ_{c}-related cytokine receptors rescues CD4-mediated inhibition of T cell proliferation.

FIG. 2A is a histogram showing that stimulation of purified CD4⁺ T lymphocytes through γ_{c}-related cytokine receptors rescues CD4-mediated inhibition of TCR/CD3 induced IL-2R expression. FIG. 2B is a bar graph showing that stimulation of purified CD4⁺ T lymphocytes through γ_{c}-related cytokine receptors rescues CD4-mediated inhibition of TCR/CD3 induced IL-2R expression.

FIG. 3A is a scanned image of a Western blot which demonstrates that ligation of purified CD4⁺ T lymphocytes with gp120 or anti-CD4 inhibits TCR/CD3 induced JAK3 expression and activation. FIG. 3B is a bar graph showing that purified CD4⁺ T cells incubated with gp120 or anti-CD4 for 48 hrs have reduced TCR/CD3-induced JAK3 expression and activation.

FIG. 4 is a scanned image of a Western blot showing CD4 mediated inhibition of T cell activation correlates with inhibition of JAK3 expression.

FIG. 5A is a bar graph illustrating that CD4 priming does not inhibit TCR/CD3 induced JAK1 activation. FIG. 5B is a scanned image of a Western blot illustrating that CD4 priming does not inhibit TCR/CD3 induced JAK1 activation.

FIG. 6A is a scanned image of a Western blot showing that TCR/CD3-induced JAK3 expression is inhibited in HIV-infected CD4⁺ T lymphocytes. FIG. 6B is a bar graph illustrating that TCR/CD3-induced JAK3 expression is inhibited in HIV-infected CD4⁺ T lymphocytes.

FIG. 7 is a bar graph illustrating that JAK3 expression is inhibited in TCR/CD3-stimulated T lymphocytes from HIV infected patients.

FIG. 8A is a scanned image of a Western blot showing that JAK3 expression is inhibited in TCR/CD3-stimulated HIV infected T lymphocytes, and that IL-7 increases JAK3 expression in these lymphocytes. FIG. 8B is a bar graph showing that JAK3 expression is inhibited in TCR/CD3-stimulated HIV-infected T lymphocytes, and that IL-7 increases JAK3 expression in these lymphocytes.

FIG. 9A is a scanned image of a Western blot showing that HIV-1 infection of CD4⁺ T lymphocytes inhibits activation of JAK3. FIG. 9B is a scanned image of a Western blot showing that HIV-1 infection of CD4⁺ T lymphocytes inhibits JAK3 kinase activity.

FIG. 10 is a scanned image of a Western blot that demonstrates that T lymphocytes from an HIV-infected patient show complete inhibition of T cell activation-induced JAK3 kinase activity, and that IL-2 restores JAK3 kinase activity in these lymphocytes.

FIG. 11A is a bar graph illustrating that JAK3 kinase activity is significantly inhibited in CD4+ T lymphocytes that are CD4-ligated prior to T cell activation, and that IL-2 increases JAK3 kinase activity in these lymphocytes. FIG. 11B is a scanned image of a Western blot showing that JAK3 kinase activity is significantly inhibited in CD4⁺ T lymphocytes that are CD4-ligated prior to T cell activation, and that IL-2 increases JAK3 kinase activity in these lymphocytes.

FIG. 12A is a graph depicting the ratio of luciferase activity in WE17/10 T cells electroporated with a STAT5-responsive luciferase vector and an expression vector encoding for an HIV-1 protein compared to cells electroporated with a STAT5-responsive luciferase vector and a control expression vector. Data are an average of 5 experiments and the error bars represent standard error of the mean (SEM). FIGS. 12B, 12C, and 12D are graphs depicting the ratio of JAK3/actin, STAT5/actin, and pSTAT5/actin, respectively, of WE17/10 cells mock infected or infected with NL4-3 or NL4-3 deleted of vpr, vpu, or nef. FIG. 12E depicts the luciferase activity measured from HeLa cells comprising vectors containing an HIV LTR driven luciferase, IL-2 receptor chains and JAK3, with or without Nef. Cells were cultured in the presence or absence of IL-2.

FIG. 13 is the sequence (SEQ ID NO: 5) of the HIV-1 3′ long terminal repeat (LTR). The three bolded sequences are the three potential STAT5 binding sites (S1, S2, and S3).

FIG. 14 is an EMSA (ElectroMobility Shift Assay) of oligonucleotides representing the proposed HIV-1 3′ LTR STAT5 binding sites S1 (lanes 1-3) and S3 (lanes 4-6) and the positive control of the consensus STAT5 binding site within the Bcl-XL gene promoter (lanes 7 and 8). The oligonucleotides were labeled with ³²P, incubated with WE17/10 nuclear extracts and optionally 50-fold excess of unlabeled oligonucleotide (lanes 2, 5, and 7) or 100-fold excess of unlabeled oligonucleotide (lanes 3 and 6). Migration of STAT5 and proposed STAT5 tetramer containing constructs are indicated at the right.

FIG. 15 is an EMSA of ³²P labeled oligonucleotides representing the proposed HIV-1 3′ LTR STAT5 binding sites S1 (lanes 1-3) and S3 (lanes 4-6). The oligonucleotides were incubated with WE 17/10 nuclear extracts that had been previously incubated with 0.5 μg (lanes 2 and 5) or 1.0 μg (lanes 3 and 6) of anti-STAT5 antibody. Migration of STAT5 containing constructs are indicated at the right.

FIG. 16 is an EMSA of ³²P labeled oligonucleotides representing the proposed HIV-1 3′ LTR STAT5 binding sites S1 (lanes 6-10), S2 (lanes 11-15), and the putative STAT5 binding site within the Bcl-XL gene promoter (lanes 1-5). The oligonucleotides were incubated with T cell nuclear extracts and then optionally with antibodies specific for STAT1 (lanes 2, 7, and 12), STAT3 (lanes 3, 8, and 13), the carboxy-terminus of STAT5 (lanes 4, 9, and 14), or the SH2 and SH3 domains of STAT5 (lanes 5, 10, and 15). Migration of STAT5 containing constructs and constructs additionally bound by antibody (supershifted (SS)) are indicated at the left.

FIG. 17 is a graph depicting the ratio of luciferase activity in resting CD4⁺ cells transfected with a LTR-luciferase vector and optionally with a STAT5 expression vector compared to cells transfected with a control-luciferase vector. Six hours after transfection, the cells were optionally contacted with IL-2. The cells were subsequently lysed and the luciferase activity measured.

FIG. 18A is an image of the PCR products from the ChIP (chromatin immunopreciptation) assay performed with antibodies to STAT5, NFκB, or with rabbit IgG. The “Input” sample is the PCR product of the cellular lysate not subjected to immunoprecipitation. FIG. 18B is a graph depicting the amount of DNA bound by a specific protein (STAT5 or NFκB) compared to a non-specific control, as determined by real time quantitative PCR.

FIGS. 19A, 19B, and 19C are the 5′ LTRs of human T-lymphotropic virus 1 (HTLV-1) (SEQ ID NO: 7), feline immunodeficiency virus (FIV) (SEQ ID NO: 8), and simian immunodeficiency virus (SIV) (SEQ ID NO: 9), respectively. Potential STAT5 binding sites are in bold.

FIG. 20A is the amino acid sequence of STAT5A (NM_{—}003152; SEQ ID NO: 10) and FIG. 20B is the amino acid sequence of STAT5B (NM_{—}012448; SEQ ID NO: 11).

### DETAILED DESCRIPTION OF THE INVENTION

In normal T cells, ligand binding of the γ_{c}-related cytokine receptors results in tyrosine phosphorylation, and consequent activation, of the attached JAK3. Latent cytoplasmic transcription factors termed STATs (signal transducers and activators of transcription) are recruited to the cytokine receptor and are phosphorylated by JAK3. The phosphorylated STAT's then enter the nucleus to regulate transcription of many different genes (Darnell, J. E. Jr. (1997) Science, 277:1630). Studies of genetically deficient mice and humans show that γ_{c} and JAK3 are critical for the normal development and function of the immune system (Noguchi et al. (1997) Proc. Natl. Acad. Sci. 94:11534-115349; Russell et al. (1994) Science, 266:1042; Darnell, J. E. Jr. (1997) Science, 277:1630; Cao et al. (1995) Immunity, 2:223; and Nosaka et al. (1995) Science 270: 800). In addition, crosslinking of the γ_{c} chain of the γ_{c}-related cytokine receptors, IL-2R, IL-4R or IL-7R, prevents induction of anergy in murine T cell lines that have been activated in the absence of costimulation (Boussiotis et al. (1994) Science, 266:1039).

Prior to the present invention, however, it was not known that ligation of T cells through CD4 (e.g., by envelope glycoproteins expressed by human immunodeficiency virus (HIV)) prior to activation of the T cell would inhibit TCR activation-induced action of JAK3 in T cells, or that inhibition of JAK3 action was correlated with a decrease in CD4⁺ T cell responsiveness in HIV-infected individuals. The present inventors' discovery provides the basis for a novel therapeutic approach to reverse and/or prevent the early immunodeficiency seen in HIV-infected individuals.

Specifically, therapeutic and diagnostic strategies of the present invention which selectively increase the action of JAK3 in CD4⁺ T lymphocytes of a patient with HIV infection will restore immune function to HIV-infected CD4⁺ T lymphocytes, as well as to CD4⁺ T lymphocytes which are not infected by HIV, but for which CD4 has been ligated by HIV envelope glycoprotein (i.e., bystander CD4⁺ T cells). In the case of the HIV-infected CD4⁺ T cells, inhibition of JAK3 as a result of CD4 ligation by the immunodeficiency virus suppresses activation of the T cell. Without being bound by theory, the present inventors believe that such suppression contributes to the ability of the virus to remain latent in the infected T cell and thereby escape detection by the host immune system. Increasing immune responsiveness, and particularly, activation, in HIV-infected T cells by increasing JAK3 action according to the present invention, will result in the replication of HIV within the cell and expression of viral proteins on the T cell surface. These cells can then be recognized and eliminated by the host immune response. In the case of the CD4-ligated, but non-infected CD4⁺ T cells, the restoration of immune function allows these cells to become active participants in immune surveillance and host defense, including in the immune response to HIV.

In addition, strategies targeting JAK3 action as disclosed herein are believed to be capable of contributing to the maintenance of T cell survival (i.e., preventing or inhibiting apoptosis) and restoration of T cell maturation (i.e., T cell development) in HIV disease. IL-2 prevents apoptosis of CD4⁺ T cells from HIV seropositive individuals in vitro, and this has been correlated with Bcl-2 expression (Adachi et al. (1996) LT. Inununol. 157:4184). Forced expression of Bcl-2 has been shown to restore all stages of T lymphopoiesis in γ_{c} deficient mice (Kondo et al., (1997) Immunity, 7:155). Recently, it has been shown that the apoptosis inhibition effected by IL-2 is restricted to naive T cells, whereas in activated T cells, IL-2 actually contributes to the induction of apoptosis (Abbas (1998) Immunity, 8:615-623). The present inventors have shown that by increasing the action of JAK3 in CD4⁺ T lymphocytes in HIV infected individuals, CD4⁺ T cells can become activated, a requisite phenotype for productive infection of the T cells. After allowing the cells to become activated, the increase in JAK3 induced by the method and composition of the present invention can additionally contribute to the ability of the HIV-infected cell to undergo apoptosis and be eliminated.

In view of the present inventors' discovery that JAK3 inhibition is directly correlated with a decrease in T cell responsiveness, and that increasing JAK3 action through, for example, γ_{c} chain receptors, increases T cell responsiveness, less toxic and/or more effective strategies which specifically increase action of JAK3 can now be developed which also protect naive or resting T cells from apoptosis associated with HIV-infection and facilitate reconstitution of the T cell immune system. The present inventors have provided evidence that potentially less toxic γ_{c} cytokines, selective activation of JAK3, and/or more localized therapy which targets JAK3 will provide valuable therapeutic tools for treatment of HIV infected patients. In combination with aggressive anti retroviral therapy, therapies that prevent loss of immune surveillance, survival and development could, significantly delay progression of HIV disease.

One embodiment of the present invention relates to a method to increase CD4⁺ T lymphocyte immune responsiveness in a patient having human immunodeficiency virus (HIV) infection. Such a method includes the step of increasing JAK3 action in CD4⁺ T lymphocytes of the patient, wherein the increase in JAK3 action is sufficient, to increase immune responsiveness in the CD4⁺ T lymphocytes. Preferably, the method increases JAK3 action in CD4⁺ T lymphocytes in which CD4 has been ligated by gp120. Such a method is particularly useful for: restoring immune surveillance and host defense capabilities to an HIV-infected host by increasing immune responsiveness in CD4-ligated, non-infected cells; allowing HIV-infected cells to become activated by increasing immune responsiveness which allows for expression of HIV proteins by the T cell (i.e., productive infection) and subsequent recognition/elimination of the T cell by the host immune system; and/or enhancing survival/development of CD4⁺ T lymphocytes to reconstitute effective cellular immunity in an HIV-infected host.

According to the present invention, the phrase, “T lymphocyte immune responsiveness” or “T lymphocyte responsiveness”, refers to the ability of a T lymphocyte to be activated by (e.g., respond to) antigenic and/or mitogenic stimuli which results in induction of T lymphocyte activation signal transduction pathways and activation events. As used herein, antigenic stimulation is stimulation of a T cell by binding of the T cell receptor to an MHC-peptide antigen that is specifically recognized by the T cell in the context of the appropriate costimulatory signals necessary to achieve T cell activation or by binding of the T cell receptor to a superantigen. Mitogenic stimulation is defined herein as any non-antigen stimulation of T cell activation, including by mitogens (PHA) and antibodies (anti-TCR, anti-CD3, including divalent and tetravalent antibodies), such compounds being referred to generically as T cell mitogens. According to the present invention, “T cell receptor-mediated activation” refers to either antigenic or non-antigenic T cell activation which is initiated at the level of the T cell receptor and proceeds through the T cell receptor signal transduction pathway. Both antigenic stimulation and the forms of mitogenic stimulation which act at the level of the T cell receptor (i.e., anti-TCR/CD3) result in T cell receptor mediated activation, whereas other modes of stimulation such as phorbol ester/ionomycin stimulation bypass the T cell receptor and therefore, do not induce T cell receptor-mediated activation.

T cell activation events include, but are not limited to, T cell proliferation, cytokine production, upregulation of cytokine receptors, calcium mobilization, and/or cytoskeletal reorganization. According to the present invention, the terms “T lymphocyte” and “T cell” can be used interchangeably herein. In addition, the phrases, “T lymphocyte responsiveness”, “T lymphocyte immune responsiveness”, “T lymphocyte function” and “T lymphocyte immune function” can be used interchangeably herein.

As used herein, the phrase “signal transduction pathway” refers to at least one biochemical reaction, but more commonly a series of biochemical reactions, which result from interaction of a cell with a stimulatory molecule. The interaction of an antigenic or mitogenic stimulatory molecule is with a T cell generates a “signal” that is transmitted through a T cell activation signal transduction pathway, ultimately resulting in events associated with T lymphocyte activation. T lymphocyte signal transduction pathways include signal transduction molecules, for example, cell surface receptors (eg., TCR/CD3) and intracellular signal transduction molecules, which mediate the transmission of the signal. As used herein, the phrase “cell surface receptor” includes molecules and complexes of molecules capable of receiving a signal and the transmission of such a signal across the plasma membrane of a cell. The phrase “intracellular signal transduction molecule,” as used herein, includes those molecules or complexes of molecules involved in transmitting a signal from the plasma membrane of a cell through the cytoplasm of the cell, and in some instances, into the cell's nucleus. The phrase “stimulatory molecule”, as used herein, can include ligands capable of binding to cell surface receptors to initiate a signal transduction pathway, as well as intracellular initiator molecules capable of initiating a signal transduction pathway from inside a cell.

Activation characteristics of a T lymphocyte that is responsive or has immune function include, but are not limited to: production of cytokines by the T cell (e.g., IL-2, IL-4, IL-10, IFN-γ); mobilization of intracellular and/or extracellular calcium; T cell proliferation; upregulation of cytokine receptors on the T cell surface, including IL-2R; upregulation of other receptors associated with T cell activation on the T cell surface; reorganization of the cytoskeleton; upregulation of expression and activity of signal transduction proteins associated with T cell is activation; and/or induction of cytolytic activity. A T lymphocyte that is responsive or has immune function, when activated, preferably is capable of proliferating and/or producing one or more cytokines. In addition, such a T lymphocyte is preferably capable of upregulating IL-2 receptors (IL-2R) on the cell surface. Even more preferably, a responsive T lymphocyte, when activated, is capable of performing T lymphocyte effector functions, such as providing help to B lymphocytes, secreting immunoregulatory cytokines and/or engaging in cytolytic activity.

The ability of a T lymphocyte to respond, or become activated, by an antigenic or mitogenic stimulus can be measured by any suitable method of measuring T cell activation. Such methods are well known to those of skill in the art. For example, after a T cell has been stimulated with an antigenic or mitogenic stimulus, characteristics of T cell activation can be determined by a method including, but not limited to: measuring the amount of IL-2 produced by a T cell (e.g., by immunoassay or biological assay); measuring the amount of other cytokines produced by the T cell (e.g., by immunoassay or biological assay); measuring intracellular and/or extracellular calcium mobilization (e.g., by calcium mobilization assays); measuring T cell proliferation (e.g., by proliferation assays such as radioisotope incorporation); measuring upregulation of cytokine receptors on the T cell surface, including IL-2R (e.g., by flow cytometry, immunofluorescence assays, immunoblots); measuring upregulation of other receptors associated with T cell activation on the T cell surface (e.g., by flow cytometry, immunofluorescence assayst immunoblots); measuring reorganization of the cytoskeleton (e.g., by immunofluorescence assays, immunoprecipitation, immunoblots); measuring upregulation of expression and activity of signal transduction proteins associated with T cell activation (e.g., by kinase assays, phosphorylation assays, immunoblots, RNA assays); and measuring specific effector functions of the T cell (e.g., by proliferation assays, cytotoxicity assays, B cell assays). Methods for performing each of these measurements are well known to those of ordinary skill in the art, and all such methods are encompassed by the present invention.

The phrases “decrease in T lymphocyte responsiveness,” “decrease in T lymphocyte immune function” and “T lymphocyte unresponsiveness” refer to any measurable reduction (i.e., decrease, downregulation, inhibition) in any characteristic of T lymphocyte immune responsiveness as defined above, as compared to a control T lymphocyte which is responsive (i.e., has immune function, can be activated) to antigenic or mitogenic stimuli. One type of T cell unresponsiveness can be referred to as “anergy”, which is typically used to refer to a T cell in which reactivity (i.e., response) to an antigenic or mitogenic stimulus is diminished to the point that substantially no measurable immune response is observed. A T cell that is undergoing or has undergone apoptosis, or programmed cell death, is also included in the definition of T lymphocyte unresponsiveness as used herein.

A “control” T lymphocyte is defined as a T lymphocyte in which a parameter to be evaluated (e.g., TCR-induced proliferation) is intentionally maintained, induced or inhibited, and/or in which the measurement of the parameter in the control cell is specifically designated to serve as a base-line measurement against which another cell (a test cell) is to be evaluated. Preferably, the control is substantially genetically similar (i.e., from the same species or source) or identical (i.e., clonal) to the T lymphocyte to be evaluated. T cells from a patient infected with HIV can be evaluated as compared to T cells from a control patient(s) who is not infected with HIV, or as compared to a non-infected human T cell line or clone, for example. Control patients preferably are selected to be similar to the test patient in characteristics such as age and/or gender in order to minimize the effect of such factors on the immune response.

An increase (i.e., improvement, upregulation, restoration or rescue) in T lymphocyte immune responsiveness or function is defined herein as any measurable increase (i.e., induction, upregulation) in any characteristic of T lymphocyte immune responsiveness as defined above, as compared to the same characteristic in a control lymphocyte in which a decrease in T lymphocyte responsiveness and/or a baseline level of low T cell responsiveness has previously been established, and/or as compared to a previous measurement of the responsiveness of the T lymphocyte to be evaluated prior to or at the time of employment of a method according to the present invention. Restoration or increase of immune responsiveness or function includes a measurable increase the ability of the tested T lymphocyte to display any characteristic of T lymphocyte activation and/or an increase in the survival/development of naive T cells, as well as an inhibition or prevention of apoptosis of naive or resting T cells.

T lymphocyte responsiveness or function in a patient having HIV infection can be determined, for example, by isolating a sample of T cells, and preferably CD4⁺ T cells, from the HIV-infected patient (e.g., from blood, as described in Example 1), and using any of the above described methods to evaluate the function or responsiveness of the isolated T lymphocytes in vitro, as compared to the appropriate control(s) defined above. It is to be noted that an isolated sample of T cells is a sample of T cells that has been removed from its natural milieu. As such, the term “isolated” does not necessarily reflect the extent to which the sample has been purified, such that other cell types may be present in the isolated sample. Alternatively, or in addition, T lymphocyte responsiveness or function in a patient having HIV infection can be determined by tests which are correlated with T lymphocyte function in vivo, as compared to the appropriate controls. Such tests include, but are not limited to delayed hypersensitivity reaction (DTH) testing. DTH reactions are indicative of a local cellular immune response against a defined antigen and the tests are typically performed by injecting a small amount of a defined antigen, such as tuberculin, into the skin, and evaluating the level of inflammatory response to the antigen at the site of injection. Such in vivo evaluations of cellular immunity are routinely performed by those of skill in the art.

As used herein, the term “human immunodeficiency virus” or “HIV” can refer to any strain of HIV, including both HIV-1 and HIV-2. According to the present invention, an HIV infected CD4⁺ T lymphocyte is defined as a T lymphocyte for which at least one CD4 molecule on the surface of the T lymphocyte has been ligated by an envelope glycoprotein of at least one human immunodeficiency virus particle, wherein the virus has entered the T cell. An infected T cell can be productively infected (i.e., the virus is active and replicating) or latently infected (i.e., the virus is dormant and not replicating). A CD4⁺ T lymphocyte that expresses CD4 that has been ligated by gp120 (or artificially by anti-CD4, for example) can also be referred to as a “CD4-ligated” or “CD4-contacted” T cell, and includes both HIV-infected and uninfected (i.e., non-infected) CD4⁺ T lymphocytes. It is known that CD4 on uninfected T cells can be contacted by gp120 that is expressed by HIV, shed by HIV, or expressed by a productively infected cell (i.e., which expresses gp120 on its surface)—and the result is reduction in immune responsiveness of the contacted CD4⁺ T cell, even in the absence of subsequent HIV infection of the cell. A CD4-ligated T cell for which the method of the present invention is useful for increasing immune function is a CD4-ligated T cell in which CD4 was ligated in the absence of T cell receptor-mediated antigenic or mitogenic activation of the T cell.

As used herein, the term “latent reservoir” refers to cells or sites in a host that are latently infected with a microbe.

As used herein, the term “structured treatment interruption” refers to the cessation of treatment of a patient for a microbial infection. Typically, structured treatment interruptions are supervised withdrawals of anti-microbial drug therapy which are followed by reinitiation of anti-microbial drug therapy. Structured treatment interruptions may allow for autologous microbe levels to increase during the drug withdrawal period.

The methods and compositions of the present invention are suitable for use in any patient with an HIV infection. In particular, the present methods and compositions are suitable for use in any HIV-infected patient in which there is a reasonable likelihood that a therapeutic benefit can be obtained by the use of such method or composition. Such a patient can be characterized as having a sufficient number of “rescueable CD4⁺ T cells” such that increasing immune responsiveness in these T lymphocytes by the method or composition of the present invention would be reasonably expected to provide a measurable benefit to the patient, alone or in combination with other HIV therapies. As used herein, a “rescueable T lymphocyte” is a T lymphocyte with reduced immune responsiveness in which JAK3 action can be increased by the method or composition of the present invention, such increase being sufficient to increase immune responsiveness in the T lymphocyte.

More particularly, an HIV-infected patient in which the method and composition of the present invention are suitable for use can be identified by isolating a sample of T lymphocytes, and preferably CD4⁺ T lymphocytes from the patient, and determining whether the T lymphocytes, when activated in vitro (e.g., by T cell receptor-mediated activation such as antibody or mixed lymphocyte reaction), shows a statistically significant (p<0.05) increase in JAK3 action when contacted with a compound that regulates JAK3 action (e.g., a cytokine selected from IL-2, IL-4, IL-7, IL-9, IL-13 or IL-15, or other compounds as disclosed herein), as compared to a control sample of T lymphocytes isolated from the same patient that are activated but not cultured with the compound. Using such an in vitro test, a candidate patient can be evaluated to determine whether the T cells in the patient are likely to respond to treatment with the compound, and additionally, whether one type of compound might work better than another in the patient. For example, if the compound is a cytokine that binds to a γ_{c} receptor, T cells from a given patient may show a marginal increase in T cell responsiveness and JAK3 action when contacted with IL-2, but show a significant increase in T cell responsiveness and JAK3 action when contacted with IL-7. Such a patient would therefore not be a suitable candidate for IL-2 therapy, but a good candidate for IL-7 therapy. As discussed in detail herein, an increase in JAK3 action can be measured by any suitable method, including, but not limited to: measurement of JAK3 transcription (i.e., determining JAK3 mRNA levels), measurement of JAK3 translation (determining JAK3 protein levels, e.g., by flow cytometry, immunoblot or other appropriate technique), measurement of phosphorylation of JAK3, measurement of JAK3 enzymatic activity (e.g., kinase activity/phosphorylation of a substrate, including JAK3 phosphorylation of STAT5), measurement of JAK3 protein binding activity (e.g. binding or association with a STAT protein or to a γ_{c}-bearing receptor), measurement of JAK3 protein translocation within a cell and/or measurement of other biological events associated with the JAK3 signal transduction pathway (e.g., measurement of transcriptional regulation of genes by STATs that associate with JAK3).

In another embodiment, a suitable HIV-infected candidate for treatment using the present method and composition can be characterized in that in a sample of T lymphocytes isolated from the patient, when activated in vitro (e.g., by T cell receptor-mediated stimulation), show a measurable increase of at least about 10%, and preferably at least about 25%, and more preferably at least about 50%, and more preferably at least about 75% in any measure of T cell responsiveness/activation as discussed above when cultured with a compound that targets JAK3 action as disclosed herein, as compared to a control T cell cultured in the absence of such a compound. Such measure of T cell activation can include, but is not limited to JAK3 action, T cell proliferation, cytokine production, calcium mobilization, and/or effector function, as compared to a control sample of T lymphocytes isolated from the same patient that are activated but not cultured with the compound.

A measurable benefit to a patient in which the method of the present invention has been employed can be determined, without limitation, by one or more of: (1) measurable maintenance of T lymphocyte survival (e.g., less than about 50%, and more preferably, less than about 25%, and more preferably, less than about 10%, and even more preferably, less than about 5% loss in blood CD4⁺ T lymphocyte number after employing the present method as compared to an average CD4⁺ T lymphocyte loss calculated in untreated HIV infected patients); (2) any measurable increase in CD4⁺ T lymphocyte numbers (e.g., at least about 5%, and preferably, at least about 10%, and more preferably at least about 25%, and even more preferably at least about 50% increase in blood CD4⁺ T lymphocyte numbers after employing the present method); (3) measurable increase in CD4⁺ T lymphocyte function, as measured by any of the above-described in vitro methods, after employing the present method; (4) measurable increase in anti-HIV immune responses (e.g., as measured by numbers of antibodies or cytotoxic T cells directed against HIV epitopes) after employing the present method; (5) measurable inhibition of significant increases in viral load (e.g., viral load increases are no more than about 50%, and preferably no more than about 25%, and more preferably no more than about 10%, and even more preferably no more than about 5% of the initially measured level prior to treatment) after employing the present method; (6) maintenance of normal immune responses to foreign agents in vivo (e.g., as measured by DTH reactions, lack of development of opportunistic infections) after employing the present method; and (7) increase of normal immune responses to foreign agents in vivo (e.g., as measured by DTH reactions) after employing the present method.

These measures of benefit to a patient in which the method of the present invention has been employed are typically measured over the period of time during which the treatment is continuing to be employed, which may be for extensive periods, until viral load is no longer detectable in the patient, or for the lifetime of the patient.

One aspect of the present invention is directed to a method for increasing CD4⁺ T lymphocyte immune responsiveness in a patient who has early-onset HIV infection. The present inventors have surprisingly discovered that an early window of opportunity exists for rescue (i.e., restoration) of T cell immune responsiveness by increasing JAK3 action. More particularly, the present inventors have found that JAK3 action is significantly inhibited in CD4 primed T cells at both 24 hours and 48 hours after T cell activation. Although an increase in JAK3 action naturally occurs at 72 hours after T cell activation (i.e., in the absence of intervention as described herein), which correlates with an increase in IL-2R expression, the T cells still fail to proliferate in response to TCR stimulation (i.e., are unresponsive) (See Examples 1 and 3). Moreover, at time points later than between 24 and 48 hours after T cell activation, the present inventors have found that the T cells can not be rescued in vitro by increasing the action of JAK3 through the administration of cytokines to the cell (See Example 3).

Since early onset patients typically have a greater number of CD4⁺ T cells to be treated, and, due to lack of progression of the disease and opportunistic infections following therefrom, can typically also withstand greater stress and toxicity which may accompany therapeutic treatments, such patients may respond better to the method of the present invention (or at lower doses of a composition/compound according to the present invention), than patients in which the HIV infection has advanced. It is to be understood, however, that the present method and composition are useful for treating any HIV-infected patient which may derive a benefit from such therapy as discussed above.

Specifically, a patient with early-onset HIV infection who is a suitable candidate for the method of the present invention can be defined herein as a patient that meets one or more of the following criteria: (1) the patient has a blood CD4⁺ T cell count of at least about 100 cells/mm³, and preferably, at least about 200 cells/mm³, and more preferably, at least about 300 cells/mm³, and even more preferably, at least about 400 cells/mm³ as determined within 30 days of the time of employment of the present method; (2) the patient has an HIV serum load of less than about 400 copies/ml, and preferably, less than about 300 copies/ml, and more preferably, less than about 200 copies/ml, and even more preferably, less than about 100 copies/ml, and most preferably undetectable viral load, as determined by plasma RNA PCT within 30 days of when the method is employed.

As used herein, the phrase “JAK3 action” refers to the expression of JAK3 (i.e., transcription and/or translation) and/or any biological activity (i.e., function(s)) exhibited or performed by a naturally occurring form of JAK3 as measured or observed in vivo (i.e., in the natural physiological environment of the protein) or in vitro (i.e., under laboratory conditions). For example, JAK3 action can include, but is not limited to, JAK3 transcription, JAK3 translation, phosphorylation of JAK3, JAK3 enzymatic activity (e.g., kinase activity, including JAK3 phosphorylation of STATs), JAK3 protein-binding activity (e.g., to a STAT protein or to a γ_{c} bearing receptor), JAK3 protein translocation within a cell and/or biological events associated with the JAK3 signal transduction pathway (e.g., transcriptional regulation of genes by STATs that associate with JAK3, (Darnell, J. E. Jr. (1997) Science 277:1630). An increase in JAK3 action, including an increase in JAK3 expression or an increase in the biological activity of JAK3, can also be referred to as amplification, overproduction, activation, enhancement, up-regulation or increased action of JAK3. An increase in JAK3 action is any measurable increase in JAK3 action in a cell as compared to a control cell in which JAK3 action is intentionally maintained, and/or in which the level of JAK3 action in the control cell is specifically designated to serve as a base-line measurement. Similarly, a decrease in JAK3 action, including a decrease in JAK3 expression or a decrease in the biological activity of JAK3, can also be referred to as inactivation (complete or partial), down-regulation, or reduced or diminished action of JAK3. A decrease in JAK3 action is any measurable decrease in JAK3 action in a cell as compared to a control cell in which JAK3 action is intentionally maintained, and/or in which a level of JAK3 action in the control cell is specifically designated to serve as a base-line measurement.

In one embodiment of the present invention, the method of increasing JAK3 action in a CD4⁺ T lymphocyte of an HIV infected patient is employed in conjunction with the administration to the patient of one or more anti-retroviral therapeutic compounds. Such compounds include any compound that is useful for inhibiting or destroying retroviruses such as HIV in a patient. Such compounds include, but are not limited to, inhibitors of reverse transcriptase, protease inhibitors, attenuated virus and viral protein vaccines, inhibitors of HIV gene expression, and/or antibodies or synthetic molecules that block CD4 or chemokine receptors. Currently, the most widely used of such compounds include, but are not limited to AZT, ddI, ddC, d4T, 3TC and protease inhibitors.

In one embodiment of the present method, JAK3 action is increased in CD4⁺ T lymphocytes by administering to the CD4⁺ T lymphocytes of the HIV-infected patient a composition that contains at least one compound that increases the action of JAK3 in the CD4⁺ T lymphocytes, and particularly in the CD4-ligated T lymphocytes, including in both HIV-infected and uninfected CD4⁺ T lymphocytes.

It is to be noted that the term “a” or “an” entity refers to one or more of that entity; for example, a compound refers to one or more compounds, or to at least one compound. As such, the terms “a” (or “an”), “one or more” and “at least one” can be used interchangeably herein. It is also to be noted that the terms “comprising”, “including”, and “having” can be used interchangeably.

One class of compounds that is suitable for use in such a composition includes compounds that selectively bind to and stimulate a T cell surface receptor having a γ_{c} chain. Such a receptor includes, but is not limited to, the interleukin-2 receptor (IL-2R), IL-4R, IL-7R, IL-9R, IL-13R and IL-15R. As such, suitable compounds for use in the composition to be administered according to the present method include IL-2, IL-4, IL-7, IL-9, IL-13 and/or IL-15. In a preferred embodiment, the cytokines IL-7, IL-9, IL-13 and/or IL-15 are administered to the patient. These cytokines have the advantage of providing the desired effect of increasing the action of JAK3, and may potentially have less toxic side effects than those caused by IL-2 or IL-4, which may have more pronounced and global effects on the immune system. The present invention does not preclude the use of IL-2 or IL-4, however, since such cytokines can now be used safely and effectively, given the discovery by the present inventors and the guidance for using such cytokines as provided herein. Specifically, by targeting and/or selective expression of such cytokines at the site of CD4⁺ T lymphocytes, by selecting suitable patient candidates, and/or by using administration protocols as disclosed herein, unexpected advantages are obtained for the use of such cytokines in a safe and effective manner. In addition, the present inventors' discovery allows a physician to initially screen a given patient to evaluate whether one cytokine or other compound as discussed in detail below, will be predicted to provide a better therapeutic effect in that patient (i.e., the therapy can be tailored to suit the responsiveness of the patient, since a variety of compounds having the same end effect can now be evaluated).

According to the present invention, a cytokine that is suitable for use in a composition of the present invention includes full-length cytokines, a biologically active fragment of a cytokine, a homologue of the cytokine protein, or a fusion protein in which a biologically active fragment of a cytokine is attached to one or more fusion segments. As such, reference to a given cytokine is intended to encompass all such forms of the given cytokine. As used herein, “a biologically active fragment of a cytokine” refers to a fragment (i.e., a truncated version of the full-length protein) of a cytokine protein having cytokine activity and being capable of binding to a cytokine receptor. As used herein, a homologue of a cytokine is a protein having an amino acid sequence that is sufficiently similar to a natural cytokine amino acid sequence so as to have cytokine activity (i.e. activity associated with naturally occurring, or wild type cytokines).

Suitable fusion segments for use in a fusion protein include, but are not limited to, segments that can: enhance a protein's stability; enhance the biological activity of a protein; and/or assist purification of a protein (e.g., by affinity chromatography). A suitable fusion segment can be a domain of any size that has the desired function (e.g., imparts increased stability, imparts enhanced biological activity to a protein, and/or simplifies purification of a protein). Fusion segments can be joined to amino and/or carboxyl termini of the cytokine protein-containing portion, for example and can be susceptible to cleavage in order to enable straight-forward recovery of the fusion protein, if such recovery is desired. Fusion proteins are preferably produced by culturing a recombinant cell transformed with a fusion nucleic acid molecule that encodes a protein including the fusion segment attached to either the carboxyl and/or amino terminal end of the domain containing the desired protein (e.g., cytokine-containing domain).

A preferred dose of cytokine to administer to a patient in a method of the present invention is typically from about 1×10⁶ IU/m²/day to about 12×10⁶ IU/m²/day, and more preferably, from about 1×10⁶ IU/m²/day to about 8×10⁶ IU/m²/day, and even more preferably, from about 1×10⁶ IU/m²/day to about 6×10⁶ IU/m²/day. Such a dose can be administered, for example, systemically by continual infusion for 5 days, repeated every 8 weeks. It is within the ability of one of ordinary skill in the art to determine and modify such an administration protocol according to patient improvement or decline and/or toxicity. Using pharmaceutically acceptable delivery vehicles and other routes of administration as described in detail below, and particularly by using targeting delivery vehicles, doses can be reduced.

Another compound that selectively binds to and stimulates a T cell surface receptor having a γ_{c} chain is an antibody, or ligand binding portion thereof, which selectively binds to and activates the γ_{c}-receptor. Antibodies useful in the present invention can be either polyclonal or monoclonal antibodies. Such antibodies include functional equivalents such as antibody fragments and genetically-engineered antibodies, including single chain antibodies, that are capable of selectively binding to at least one of the epitopes of the protein or mimetope used to obtain the antibodies. Antibodies of the present invention can include chimeric antibodies in which at least a portion of the heavy chain and/or light chain of an antibody is replaced with a corresponding portion from a different antibody or protein.

Generally, in the production of an antibody, a suitable experimental animal, such as a rabbit, hamster, guinea pig or mouse, is exposed to an antigen against which an antibody is desired (e.g., a γ_{c}-receptor). Typically, an animal is immunized with an effective amount of antigen that is injected into the animal. An effective amount of antigen refers to an amount needed to induce antibody reduction by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies. Such serum is useful as a reagent. Polyclonal antibodies can be further purified from the serum by, for example, treating the serum with ammonium sulfate. In order to obtain monoclonal antibodies, the immunized animal is sacrificed and B lymphocytes are recovered from the spleen. The B lymphocytes are then fused with myeloma cells to obtain a population of hybridoma cells capable of continual growth in suitable culture medium. Hybridomas producing a desired antibody are selected by testing the ability of an antibody produced by a hybridoma to bind to the antigen.

In another embodiment, a composition for use in the method of the present invention can include a compound that selectively increases the expression of JAK3 in CD4⁺ T lymphocytes by associating with (i.e., binding to) a transcription control sequence of a gene encoding JAK3, or with a translation control sequence of a mRNA transcript encoding JAK3 such that JAK3 transcription or translation, respectively, is initiated or increased in the cell. According to the present invention, a gene encoding JAK3 includes all nucleic acid sequences related to a JAK3 gene such as regulatory regions that control production of JAK3 (such as, but not limited to, transcription, translation or post-translation control regions) as well as the coding region itself. A transcription control sequence is a sequence which controls the initiation, elongation, and/or termination of transcription of a gene. Particularly important transcription control sequences are those which control transcription initiation, such as promoter, enhancer, operator and repressor sequences. Similarly, a translation control sequence is a sequence which controls the initiation, elongation, and/or termination of translation of a protein from the nucleic acid sequence comprising the transcript (i.e., mRNA).

A compound suitable for use in the method of the present invention includes an isolated, naturally occurring transcription control factor, or a homologue thereof, that selectively associates with (i.e., selectively binds to) a transcription control sequence of a gene encoding JAK3 such that transcription is initiated or increased. The complete nucleic acid sequence encoding JAK3, including portions of the untranslated regions of the gene, and the amino acid sequence of JAK3 are known and disclosed in U.S. Pat. No. 5,705,625 to Civin et al., which is incorporated herein by reference in its entirety. Initiation of transcription of JAK3 in a cell can be measured by any method of evaluating transcription known in the art, including by Northern blot analysis. Notably, several transcription factor binding sites within the JAK3 promoter have been identified (Aringer, M., et al., (2003) J. Immunol., 170:6057-64).

According to the present invention, a homologue of a protein differs from that protein by deletion (e.g., a truncated version of the protein, such as a peptide), insertion, inversion, substitution and/or derivatization (e.g., by glycosylation, phosphorylation, acetylation, myristoylation, prenylation, palmitation, amidation and/or addition of glycosylphosphatidyl inositol) of one or a few amino acid residues in the protein, whereby such modifications do not interfere with the ability of the homologue to perform the biological function of the naturally occurring protein (i.e., bind to a transcription control sequence and initiate transcription).

According to the present invention, “selective” or “selectively” as used in phrases such as “selectively binds”, “selectively activates”, “selectively increases”, and other such phrases, is defined as: to discern, discriminate, or distinguish one entity from another. For example, a compound that selectively binds to a given receptor specifically recognizes and binds to that receptor but does not recognize or bind to a different receptor. Similarly, a compound that selectively increases the action of JAK3, for example, is capable of specifically causing an increase in JAK3 and/or molecules and signal transduction pathways related to JAK3 action, without increasing the action of molecules or signal transduction pathways that are unrelated to the action of JAK3.

In another embodiment, a compound suitable for use in the method of the present invention includes a recombinant nucleic acid molecule comprising an isolated nucleic acid sequence encoding a biologically active JAK3 protein. The isolated nucleic acid sequence is operatively linked to a transcription control sequence such that the recombinant nucleic acid molecule, when transfected into a suitable host cell (i.e., a CD4⁺ T lymphocyte or a precursor cell thereof), expresses biologically active JAK3 protein. As used herein, a biologically active JAK3 protein includes a full-length JAK3 protein and homologues of JAK3, such as a JAK3 protein in which amino acids have been deleted (e.g., a truncated version of the protein, such as a peptide or fragment), inserted, inverted, substituted and/or derivatized (e.g., by glycosylation, phosphorylation, acetylation, myristylation, prenylation, palmitoylation, amidation and/or addition of glycerophosphatidyl inositol), wherein the homologue maintains the biological functions of a naturally occurring, full-length JAK3 protein. A suitable host cell for expression of a biologically active JAK3 protein according to the present method is a CD4⁺ T lymphocyte precursor (e.g., a stem cell) or a CD4⁺ T lymphocyte, and preferably, a CD4⁺ T lymphocyte precursor or a CD4⁺ T lymphocyte in an HIV-infected patient, and even more preferably, a CD4-ligated T lymphocyte in an HIV-infected patient.

According to the present invention, an isolated, or biologically pure, nucleic acid molecule or nucleic acid sequence, is a nucleic acid molecule or sequence that has been removed from its natural milieu. As such, “isolated” and “biologically pure” do not necessarily reflect the extent to which the nucleic acid molecule has been purified. An isolated nucleic acid molecule useful in the present method can include DNA, RNA, or derivatives of either DNA or RNA. An isolated nucleic acid molecule useful in the present method can include nucleic acid sequences that encode a full-length protein or a biologically active fragment thereof, and nucleic acid molecules that comprise regulatory regions.

An isolated nucleic acid molecule can be obtained from its natural source, either as an entire (i.e., complete) gene or a portion thereof capable of encoding a protein, such as a JAK3 protein or a JAK3 transcription factor, or a biologically active fragment thereof. A nucleic acid molecule can also be produced using recombinant DNA technology (e.g., polymerase chain reaction (PCR) amplification, cloning) or chemical synthesis. Nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode a JAK3 protein useful in the method of the present invention.

A nucleic acid molecule homologue can be produced using a number of methods known to those skilled in the art (see, for example, Sambrook et al., Molecular Cloning: A Laboratory Manual, Cold Spring Harbor Labs Press, 1989; and Ausubel et al., eds. (1995) Current Protocols in Molecular Biology, John Wiley and Sons, Inc.), which is incorporated herein by reference in its entirety. For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., JAK3 expression or biological activity). Techniques for screening for expression and biological activity of JAK3 are known to those of skill in the art and are described, for example in U.S. Pat. No. 5,705,625 and in the Examples section. Such techniques include, but are not limited to, RNA detection assays, immunoblots, kinase assays, and phosphorylation assays.

According to the present invention, a recombinant nucleic acid molecule encoding a given protein includes a nucleic acid sequence encoding the protein (e.g., JAK3.) or a biologically active fragment thereof operatively linked to one or more transcription control sequences. The phrase “operatively linked” refers to linking a nucleic acid molecule to a transcription control sequence in a manner such that the molecule is able to be expressed when transfected (e.g., transformed, transduced) into a host cell. Recombinant molecules can also contain additional regulatory sequences, is such as translation regulatory sequences and other regulatory sequences that are compatible with the host cell.

A recombinant molecule can be used to produce an encoded product (e.g., JAK3) useful in the method of the present invention. In one embodiment, an encoded product is produced by expressing a nucleic acid molecule as described herein under conditions effective to produce the protein. Such conditions include both ex vivo and in vivo conditions. Effective ex vivo culture conditions include, but are not limited to, effective media, bioreactor, temperature, pH and oxygen conditions that permit protein production. An effective medium refers to any medium in which a cell is cultured to produce a protein (e.g., a JAK3 protein) according to the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art. Effective in vivo conditions are normal physiological conditions at the sites where CD4⁺ T lymphocytes reside in a patient. A preferred method to produce an encoded protein is by transfecting a host cell with one or more recombinant molecules to form a recombinant cell.

Suitable host cells to transfect include any human CD4⁺ T lymphocyte precursor cell or CD4⁺ T lymphocyte. According to the method of the present invention, the host cell is preferably transfected in vivo as a result of delivery of the recombinant nucleic acid molecule to the host cell as described in detail below. The host cell can also be transfected ex vivo by removing host cells (e.g., bone marrow stem cells, or T lymphocytes from the blood of a patient which can be further purified to select CD4⁺ T lymphocytes (See Example 1)), transfecting the cells with the recombinant nucleic acid molecule, and reintroducing the cells to the host patient. Administration of a recombinant nucleic acid molecule encoding JAK3 to CD4⁺ T lymphocytes in an HIV infected patient results in expression of the nucleic acid sequence encoding JAK3 in the CD4⁺ T lymphocytes.

It may be appreciated by one skilled in the art that use of recombinant DNA technologies can improve expression of transfected nucleic acid molecules by manipulating, for example, the duration of expression of the transgene (i.e., recombinant nucleic acid molecule), the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules include, but are not limited to, operatively linking nucleic acid molecules to high-copy number plasmids, integration of the nucleic acid molecules into one or more host cell chromosomes, addition of vector stability sequences to plasmids, increasing the duration of expression of the recombinant molecule, substitutions or modifications of transcription control signals (e.g., promoters, operators, enhancers), substitutions or modifications of translational control signals (e.g., ribosome binding sites, Shine-Dalgarno sequences), and deletion of sequences that destabilize transcripts. The activity of an expressed recombinant JAK3 protein useful in the method of the present invention may be improved by fragmenting, modifying, or derivatizing nucleic acid molecules encoding such a protein.

In another embodiment, a compound suitable for use in the method of the present invention includes a JAK3 protein or another protein to be delivered intracellularly to a suitable host cell, which is operatively linked to an N-terminal protein transduction domain from HIV TAT. The HIV TAT construct for use in such a protein is described in detail in Vocero-Akbani et al. (1999) Nature Med., 5:23-33, incorporated herein by reference in its entirety. In Vocero-Akbani et al., a zymogen caspase-3 protein having endogenous cleavage sites substituted with HIV proteolytic cleavage sites was engineered as a fusion protein with an N-terminal protein transduction domain from HIV TAT. The resulting fusion protein, TAT-Casp3 transduces all cells with nearly 100% efficiency, including peripheral blood lymphocytes. Only in cells where HIV is present, however, (i.e., HIV-infected cells), is the protein cleaved by the HIV protease and the active form of the protein (i.e., caspase) released. The present invention incorporates the use of this technology, “TAT-peptide technology” to deliver proteins for use in the present method (e.g., JAK3 and other proteins which increase the action of JAK3) to cells of a recipient with nearly 100% efficiency, whereby, in one embodiment, the active form of the protein will be released only within the desired target cells. For example, the-TAT peptide construct can be engineered so that cleavage of a biologically active form of JAK3 occurs only in T lymphocytes, by the action of, for example, a T cell-specific protease which cleaves gp160 into gp120 and gp41 (described in detail in U.S. Pat. No. 5,691,183, to Franzusoff et al., incorporated herein by reference in its entirety), or in HIV infected T lymphocytes, using the HIV proteolytic sites for cleavage by an HIV protease, as described in Vocero-Akbani et al.

In one embodiment of the present invention, a composition which is administered to CD4⁺ T lymphocytes in the method of the present invention includes a pharmaceutically acceptable delivery vehicle, also referred to herein as a pharmaceutically acceptable excipient. As used herein, a pharmaceutically acceptable delivery vehicle refers to any substance suitable for delivering a composition useful in the method of the present invention to a suitable in vivo or ex vivo site. A suitable in vivo or ex vivo site is preferably a T lymphocyte, and more preferably, a CD4⁺ T lymphocyte precursor cell or a CD4⁺ T lymphocyte, and even more preferably, a CD4-ligated T lymphocyte. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, - or o-cresol, formalin and benzol alcohol. Therapeutic compositions of the present invention can be sterilized by conventional methods and/or lyophilized.

When the composition comprises a nucleic acid molecule, such as a recombinant nucleic acid molecule encoding JAK3 as discussed above, pharmaceutically acceptable delivery vehicles are preferably capable of maintaining the recombinant nucleic acid molecule in a form that, upon arrival of the nucleic acid molecule to a T cell, the nucleic acid molecule is capable of entering the T cell and being expressed by the cell.

In one embodiment of the present invention, a pharmaceutically acceptable delivery vehicle transfects or transduces multiple cell types in the recipient, but is designed to be activated only within a target cell type (e.g., a CD4⁺ T lymphocyte or an HIV-infected T lymphocyte). An example of delivery vehicles useful for such delivery include retroviral vectors, recombinant viruses or liposomes for delivery of recombinant nucleic acid molecules, and protein delivery vehicles, such as the TAT-peptide construct described above (Vocero-Akbani et al. (1999) Nat. Med., 5:23-33) for delivery of proteins. Such constructs can be designed to be selectively induced to express the desired proteins in the case of nucleic acid molecule delivery vehicles, or to release a biologically active form of the desired protein in the case of protein delivery vehicles, upon contact with a factor or protein within the target cell. Such factors/proteins include, but are not limited to, transcription/translation factors, intracellular signal transduction proteins and intracellular proteases, that are specific to the target host cell (e.g., preferably CD4⁺ T lymphocytes or precursors thereof). For example, a recombinant nucleic acid molecule expressing JAK3 can be engineered with an lck promoter that is activated selectively in CD4⁺ T cells, and not in other cell types, thereby allowing for transfection of multiple cell types with the recombinant nucleic acid molecule, but expression of the Jak3 protein only in CD4⁺ T cells. Other examples of such technology are known in the art.

In one embodiment of the present invention, a pharmaceutically acceptable delivery vehicle specifically (i.e., selectively) targets CD4⁺ T lymphocytes or precursors thereof in the HIV-infected patient. In one aspect of the invention, when elimination of HIV-infected CD4⁺ T lymphocytes is particularly desired, the delivery vehicle selectively targets HIV proteins expressed by CD4⁺ T lymphocytes. Targeting delivery vehicles of the present invention are capable of delivering a composition of the present invention to a target site in an HIV-infected patient. A “target site” refers to a site in the patient to which one desires to deliver a therapeutic composition. Preferred target sites include organs and fluids in which T lymphocytes primarily reside in a human (e.g., spleen, lymph node, blood), and more specifically, target sites are CD4⁺ T lymphocytes or precursors thereof, as previously discussed herein.

Examples of targeting delivery vehicles include, but are not limited to, artificial and natural lipid-containing delivery vehicles, retroviral vectors and antibodies. Natural lipid-containing delivery vehicles include cells and cellular membranes. Artificial lipid-containing delivery vehicles include liposomes and micelles. A lipid-containing delivery vehicle can additionally be modified to target to a particular site in an animal, thereby targeting and making use of a nucleic acid molecule or other compound of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, an antibody specific for an antigen found on the surface of a T lymphocyte, a CD4⁺ T lymphocyte or an HIV infected T lymphocyte, can be introduced to the outer surface of a liposome delivery vehicle so as to target the delivery vehicle to the cell. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics. In one embodiment of the present invention, a pharmaceutically acceptable delivery vehicle includes, but is not limited to an antibody that selectively binds to a molecule on the surface of a T lymphocyte, and preferably a CD4⁺ T lymphocyte. In one embodiment, such an antibody selectively binds to gp120. In another embodiment, a pharmaceutically acceptable delivery vehicle includes a CD4 molecule (e.g., expressed on a liposome or other vehicle, or as a soluble or hybrid molecule) which targets the vehicle to an HIV gp120 protein. In another embodiment, a pharmaceutically acceptable delivery vehicle includes an immunoliposome comprising such an antibody. An immunoliposome is a liposome which requires an antibody (conjugated to a lipid anchor) not only for specific target cell recognition but also as stabilizer of the otherwise unstable liposome (Ho et al., 1986, Biochemistry, 25: 5500-6; Ho et al. (1987) J. Biol. Chem., 262:13979-84; and Ho et al. (1987) LT Biol. Chem., 262: 13973-8; all incorporated herein by reference in their entireties).

A liposome delivery vehicle is preferably capable of remaining stable in a host patient or in an ex vivo culture for a sufficient amount of time to deliver a nucleic acid molecule or other compound according to the present invention to a preferred site in the host or culture (i.e., a CD4⁺ T cell). A liposome delivery vehicle of the present invention comprises a lipid composition that is capable of fusing with the plasma membrane of the targeted cell to deliver a nucleic acid molecule or other compound into a cell. A preferred liposome delivery vehicle of the present invention is between 5 about 100 and 500 nanometers (nm), more preferably between about 150 and 450 nm and even more preferably between about 200 and 400 nm in diameter. Suitable liposomes for use with the present invention include any liposome. Preferred liposomes of the present invention include those liposomes commonly used in, for example, gene delivery methods known to those of skill in the art. Gene delivery methods and liposomes are disclosed, for example, in U.S. Pat. No. 5,580,859, issued Dec. 3, 1996, to Felgner et al.; U.S. Pat. No. 5,589,466, issued Dec. 31, 1996, to Felgner et al.; U.S. Pat. No. 5,641,662, issued Jun. 24, 1997, all of which are incorporated herein by reference in their entirety.

Complexing a liposome with a nucleic acid molecule of the present invention can be achieved using methods standard in the art. A suitable concentration of a nucleic acid molecule of, the present invention to add to a liposome includes a concentration effective for delivering a sufficient amount of nucleic acid molecule into a host T cell such that the JAK3 protein is expressed in at a level sufficient to increase immune responsiveness in the host cell.

In another aspect of the invention, a pharmaceutically acceptable delivery vehicle includes a nucleic acid molecule of the present invention and preferably includes at least a portion of a viral genome (i.e., a viral vector), and preferably, at least a portion of a retroviral vector. Preferred viral vectors include those based on alphaviruses, poxviruses, adenoviruses, herpesviruses, and retroviruses, with those based on retroviruses, and particularly, human immunodeficiency virus being particularly preferred. Any suitable transcription control sequence can be used, including those disclosed as suitable for protein production. Particularly preferred transcription control sequences include T lymphocyte-specific transcription control sequences. The incorporation of “strong” poly(A) sequences are also preferred. Such a recombinant viral molecule can include a recombinant molecule of the present invention that is packaged in a viral coat and that can be expressed in a human after administration, referred to herein as a recombinant virus. Preferably, the recombinant virus is packaging-deficient. Methods to produce and use recombinant viral vectors and recombinant virus particles are known in the art. A particularly preferred viral vector for delivery of a recombinant nucleic acid molecule to a host cell according to the present invention is an HIV-based transducer of lymphocytes (TOL) as described in detail in Sutton et al. (1996) J. Virol., 70:7322-7326, incorporated herein by reference in its entirety.

When administered to an animal, a recombinant viral delivery vehicle as described above infects cells within the recipient and directs the production of a biologically active JAK3 protein or other protein as disclosed herein. A preferred single dose of a recombinant viral delivery vehicle of the present invention is from about 1×10⁴ to about 1×10⁷ virus plaque forming units (pfu) per kilogram body weight of the recipient.

According to the present invention, a composition which increases the action of JAK3 as described above is administered to the CD4⁺ T lymphocytes of an HIV-infected patient by any method suitable for delivering the composition to the cells. Administration routes include both in vivo and ex vivo routes. In vivo routes include, but are not limited to intradermal, intravenous, subcutaneous, oral, aerosol, intramuscular and intraperitoneal routes. Such routes can include the use of pharmaceutically acceptable delivery vehicles as described above. Ex vivo routes of administration of a composition to a culture of host cells can be accomplished by a method including, but not limited to, transfection, electroporation, microinjection, lipofection, adsorption, protoplast fusion, use of protein carrying agents, is use of ion carrying agents, and use of detergents for cell permeabilization. An effective administration protocol (i.e., administering a composition in an effective manner) comprises suitable dose parameters and modes of administration that result in increased action of JAK3 in the CD4⁺ T lymphocytes of the HIV-infected patient, preferably so that the patient experiences increased T lymphocyte immune responsiveness. Effective dose parameters can be determined using methods standard in the art. Such methods include, for example, determination of survival rates, side effects (i.e., toxicity), determination of cellular immune response effects, and progression or non-progression of HIV-related conditions. In particular, the effectiveness of dose parameters of a composition of the present invention when treating T lymphocyte unresponsiveness can be determined by assessing response rates. Such response rates refer to the percentage of treated patients in a population of patients that respond with measurable improvement in cellular immune response, and particularly, in CD4⁺ T cell immune responses.

As used herein, a resting or a naive T lymphocyte is a T lymphocyte that is not activated. A naive T lymphocyte is further defined as a T lymphocyte that has not been exposed to an antigenic or mitogenic stimulus since exiting the thymus. More particularly, a resting or naive T lymphocyte does not display the characteristics associated with activated T cells as described above (e.g., a resting T cell is not proliferating, is not producing cytokines, is not upregulating activation-associated cell surface molecules, is not capable of performing T cell effector functions, requires costimulation to become activated, etc.). The identifying characteristics of resting versus activated T cells are well known to those of skill in the art.

CD4⁺ T cells suitable for contacting using the method of the present invention can be from any suitable T cell source, and need not necessarily be a “purified” CD4⁺ T cell culture (e.g., peripheral blood mononuclear cells from an HIV-infected patient can be used). Suitable sources of CD4⁺ T cells for use in this method include, but are not limited to, T cells isolated from a human source including peripheral blood T cells, human T cell lines, human T cell clones, and human T cell hybridomas.

A CD4-ligating compound is defined herein as any compound that binds to CD4, such binding being sufficient, when the CD4 is expressed on the surface of a T cell, to transduce a signal through the CD4 molecule. According to the present invention, to be ligated at CD4, CD4 does not necessarily have to be cross-linked by the CD4-ligating compound. A CD4-ligating compound can include, but is not limited to, an antibody that binds to CD4, gp120, a fragment of gp120 sufficient to bind to CD4, a Class II major histocompatibility (MHC) molecule, a CD4 binding region of a Class II MHC molecule, a cell line expressing a recombinant Env protein and/or a human immunodeficiency virus (HIV). All such compounds are well known in the art. As used herein, a cell line expressing a recombinant Env protein is defined as any host cell (i.e., can be any cell type suitable for use in production of a recombinant protein) which has been transfected with and expresses a recombinant nucleic acid molecule encoding an Env protein (including full-length protein, fragments, derivatives and other homologues thereof) such that the Env protein is expressed on the surface of the host cell. Such cell lines are known in the art.

According to the present invention, step (a) of contacting the cell with a CD4-ligating compound can be performed, such as by mixing, under conditions in which the CD4 molecules on the surface of the CD4⁺ T cell can be bound by the CD4-ligating compound if essentially no other regulatory compounds are present that would interfere with such binding. Achieving such conditions is within the skill in the art, and includes an effective medium in which the cell can be cultured such that the cell can be ligated on CD4. Suitable culture conditions have been previously described above with reference to ex vivo culture conditions. The method, or assay, disclosed in the present invention involves contacting cells with the compound being tested for a sufficient time to allow for ligation of the CD4 on the surface of the T cells by the compound. In one embodiment, the step of contacting the T cells with a CD4-ligating compound includes exposing the T cells in the culture to human immunodeficiency virus expressing gp120 (e.g., infecting the CD4⁺ T lymphocyte with HIV).

In another embodiment, step (a) of contacting comprises the step of isolating latently HIV-infected T lymphocytes from an HIV-infected patient. In this aspect of the method, the actual step (a) of contacting the T lymphocyte with a CD4 ligating compound has occurred in vivo, by virtue of the T lymphocyte being infected with latent HIV, and the completion of step (a) is to isolate the latently infected lymphocytes from their natural milieu so that the cells can be used in the other steps (b)-(d) of the method of the present invention. Methods of isolating latently infected lymphocytes from HIV infected patients are well known in the art and are described, for example, in Chun et al. (1997) Nature 387:183-188, incorporated herein by reference in its entirety. Briefly, a sample containing T lymphocytes is isolated from an HIV infected patient. After some purification of T lymphocytes, and preferably, CD4⁺ T lymphocytes from the sample, the T lymphocytes can be identified which are not producing virus, but which either have integrated HIV in the cellular genome, or can be activated and shown to produce virus.

In step (b) of the present method, the CD4⁺ T lymphocyte, having been ligated at CD4, is contacted with a stimulatory compound that stimulates T cell receptor-mediated activation of the T lymphocyte. Suitable stimulatory compounds can include, for example, both antigenic and mitogenic stimuli as previously described herein which stimulate the T cell through the T cell receptor signal transduction pathway. Such stimulatory compounds include, but are not limited to, MHC antigen complexes, including soluble and membrane bound MHC antigen complexes, superantigens, and T cell mitogens, including PHA and antibodies (anti-TCR, anti-CD3, including divalent and tetravalent antibodies). A suitable amount of stimulatory compound to add to a cell depends upon factors such as the type of compound used (e.g., monomeric or multimeric; permeability, etc.) and the abundance of the receptor, if ligated, on a cell. Preferably, between about 1.0 nM and about 1 mM of stimulatory compound is added to a cell.

According to this method of the present invention, the cells are contacted with a putative regulatory compound that is being evaluated for its ability to increase JAK3 kinase in a manner sufficient to increase T cell responsiveness in a CD4⁺ T lymphocyte. In one embodiment, step (c) is performed prior to steps (a) or after step (a) and prior to step (b) to determine whether the putative regulatory compound is capable of preventing the induction of T cell unresponsiveness by CD4 ligation prior to T cell activation, if performed before step (a) or to assess the ability of the regulatory compound to rescue the T cell prior to T cell activation, if performed after step (a) but before step (b). In another embodiment, the step of contacting can occur after steps (a) and (b) to determine whether the compound is capable of preventing, reducing or reversing JAK3 inhibition and reduction in T cell responsiveness at time points after the stimulation of the T cell. In one embodiment of the present invention, the step of contacting the T cell with the putative regulatory compound is performed within about 24-48 hours after the step of contacting the T cell with the stimulatory compound, and more preferably, within less than about 24 hours after or before the step of contacting the T cell with the stimulatory compound.

Acceptable protocols to contact a cell with a putative regulatory compound (or a CD4-ligating or stimulatory compound) in an effective manner include the number of cells per container contacted, the concentration of putative regulatory compound(s) administered to a cell, the incubation time of the putative regulatory compound with the cell, the concentration of stimulatory compounds administered to a cell, and the incubation time of the stimulatory compounds with the cell. Determination of such protocols can be accomplished by those skilled in the art based on variables such as the size of the container, the volume of liquid in the container, the type of cell being tested and the chemical composition of the putative regulatory compound (i.e., size, charge etc.) being tested. Methods of contacting include, but are not limited to, transfection, electroporation, microinjection, lipofection, adsorption, cellular expression, protoplast fusion, use of ion carrying agents, use of protein carrying agents and use of detergents for cell permeabilization. Cellular expression can be accomplished using an expression system selected from the group of naked nucleic acid molecules, recombinant virus, retrovirus expression vectors and/or adenovirus expression vectors. Such expression systems are well known in the art and are described above.

As used herein, the term “putative” refers to compounds having an unknown regulatory activity, at least with respect to the ability of such compounds to regulate JAK3 action and CD4⁺ T cell responsiveness. Putative regulatory compounds as referred to herein include, for example, compounds that are products of rational drug design, natural products and compounds having partially defined signal transduction regulatory properties. A putative compound can be a protein based compound, a carbohydrate-based compound, a lipid-based compound, a nucleic acid-based compound, a natural organic compound, a synthetically derived organic compound, an anti idiotypic antibody, a stimulatory antibody and/or catalytic antibody, or fragments thereof. A putative regulatory compound can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See, for example, Maulik et al. (1997) Molecular Biotechnology: Therapeutic Applications and Strategies, Wiley-Liss, Inc., which is incorporated herein by reference in its entirety.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands against a desired target, and then optimize the lead molecules by either random or directed design strategies. Methods of molecular diversity are described in detail in Maulik, et al.

In a rational drug design procedure, the three dimensional structure of a regulatory compound can be analyzed by, for example, nuclear magnetic resonance (NMR) or X-ray crystallography. This three-dimensional structure can then be used to predict structures of potential compounds, such as putative regulatory compounds by, for example, computer modeling. The predicted compound structure can be used to optimize lead compounds derived, for example, by molecular diversity methods. In addition, the predicted compound structure can be produced by, for example, chemical synthesis, recombinant DNA technology, or by isolating a mimetope from a natural source (e.g., plants, animals, bacteria and fungi).

After the CD4-ligated cell has been contacted with the stimulatory compound and the putative regulatory compound, it is determined whether JAK3 action is increased in the CD4⁺ T lymphocyte. The step (d) of determining whether JAK3 action is increased can be performed by methods which include, but are not limited to, measurement of JAK3 transcription (i.e., determining JAK3 mRNA levels), measurement of JAK3 translation (determining JAK3 protein levels), measurement of phosphorylation of JAK3, measurement of JAK3 enzymatic activity (e.g., kinase activity/phosphorylation of a substrate, including STAT5), measurement of JAK3 protein binding activity (e.g. binding or association with a STAT protein or to a γ_{c}-bearing receptor), measurement of JAK3 protein translocation within a cell and/or measurement of other biological events associated with the JAK3 signal transduction pathway (e.g., measurement of transcriptional regulation of genes by STATs that associate with JAK3). Specific methods for such steps of measuring are known to those of skill in the art and are described in the Examples section, and include immunoblots, phosphorylation assays, kinase assays, immunofluorescence microscopy, RNA assays, immunoprecipitation, and other biological assays.

Yet another embodiment of the present invention relates to a method to increase CD4⁺ T lymphocyte immune responsiveness in a patient having human immunodeficiency virus (HIV) infection. Such method includes the steps of administering to the patient a composition which includes: (a) a compound selected from the group of: (1) a cytokine selected from the group of interleukin-7 (IL-7), IL-9, IL-13 and/or IL 15; (2) an antibody that selectively binds to a receptor comprising a γ_{c} chain; (3) a compound that increases the expression of JAK3 in the CD4⁺ T lymphocytes by associating with a transcription control sequence of a gene encoding the JAK3 such that JAK3 transcription is increased; (4) a JAK3 protein or biologically active fragment thereof, operatively linked to an N-terminal protein transduction domain from HIV TAT; and/or, (5) a recombinant nucleic acid molecule comprising an isolated nucleic acid sequence encoding a biologically active JAK3 protein operatively linked to a transcription control sequence. The compound is administered in an amount sufficient to increase JAK3 action in the CD4⁺ T lymphocytes. The composition additionally includes (b) one or more anti-retroviral therapeutic compounds. In one embodiment, the patient to which such a composition is administered is characterized as having a CD4⁺ T cell count of at least about 100 cells/mm³ and an HIV viral load of less than about 400 copies/ml when the method is employed. Details of such a method have been previously described in detail herein.

In any of the foregoing methods, the step of isolating a sample of T lymphocytes can be performed by any suitable protocol known in the art. Such a step is described (e.g., isolation of peripheral blood mononuclear cells), for example, in the Examples section. In step (b), suitable stimulatory compounds include any of the T cell stimulatory compounds as previously described herein, and preferably can include MHC-antigen complexes, including soluble and membrane bound MHC-antigen complexes, superantigens, and T-cell mitogens (e.g., PHA and antibodies (anti-TCR, anti-CD3, including divalent and tetravalent antibodies)). A compound suitable for binding to and activating a cytokine receptor having an γ_{c} chain have also been described above, and include, but are not limited to, cytokines that bind to γ_{c} receptors (e.g. IL-2, IL-4, IL-7, IL-9, IL-13 and IL-15) and antibodies that selectively bind to and activate γ_{c} receptors.

Methods of performing step (c) of measuring JAK3 action have been previously described herein and include, but are not limited to, measurement of JAK3 transcription (i.e., determining JAK3 mRNA levels), measurement of JAK3 translation (determining JAK3 protein levels), measurement of phosphorylation of JAK3, measurement of JAK3 enzymatic activity (e.g., kinase activity/phosphorylation of a substrate, such as STAT5), measurement of JAK3 protein binding activity (e.g. binding or association with a STAT protein or to a γ_{c}-bearing receptor), measurement of JAK3 protein translocation within a cell and/or measurement of other biological events associated with the JAK3 signal transduction pathway (e.g., measurement of transcriptional regulation of genes by STATs that associate with JAK3).

Yet another embodiment of the present invention relates to a method to eliminate latently HIV-infected T cells in an HIV-infected patient. Such a method can include an in vivo method and in vitro assay. The in vivo method includes the steps of (a) isolating a first sample of T lymphocytes from an HIV-infected patient; (b) measuring an amount of latently infected T lymphocytes in the first sample; (c) administering to the patient in vivo a composition comprising one or more compounds that increase the action of JAK3 in the CD4⁺ T lymphocytes; d) isolating a second sample of T lymphocytes from the HIV-infected patient, after step (c); (e) measuring an amount of latently infected T lymphocytes in the second sample. A decrease in the amount of latently infected CD4⁺ T lymphocytes in the second sample as compared to the amount of latently infected CD4⁺ T lymphocytes in the first sample indicates that the composition is effective to eliminate latently infected CD4⁺ T lymphocytes in the patient. Methods of isolating and measuring amounts of latently HIV-infected T cells have been previously described herein.

The in vitro method includes the steps of (a) isolating a sample of T lymphocytes from an HIV-infected patient; (b) measuring an amount of latently infected T lymphocytes in the first sample; (c) contacting the lymphocytes with a panel of compounds that bind to and activate a cytokine receptor having an γ_{c} chain; (d) identifying a compound from the panel of compounds wherein the T lymphocytes show a larger increase in JAK3 action in the presence of the compound as compared to in the presence of the other compounds in the panel and/or a larger increase in productive HIV-infection in the presence of the compound as compared to in the presence of the other compounds in the panel; and, (e) selecting and administering the compound showing the larger increase to the patient for elimination of latently HIV-infected CD4⁺ T lymphocytes in the patient.

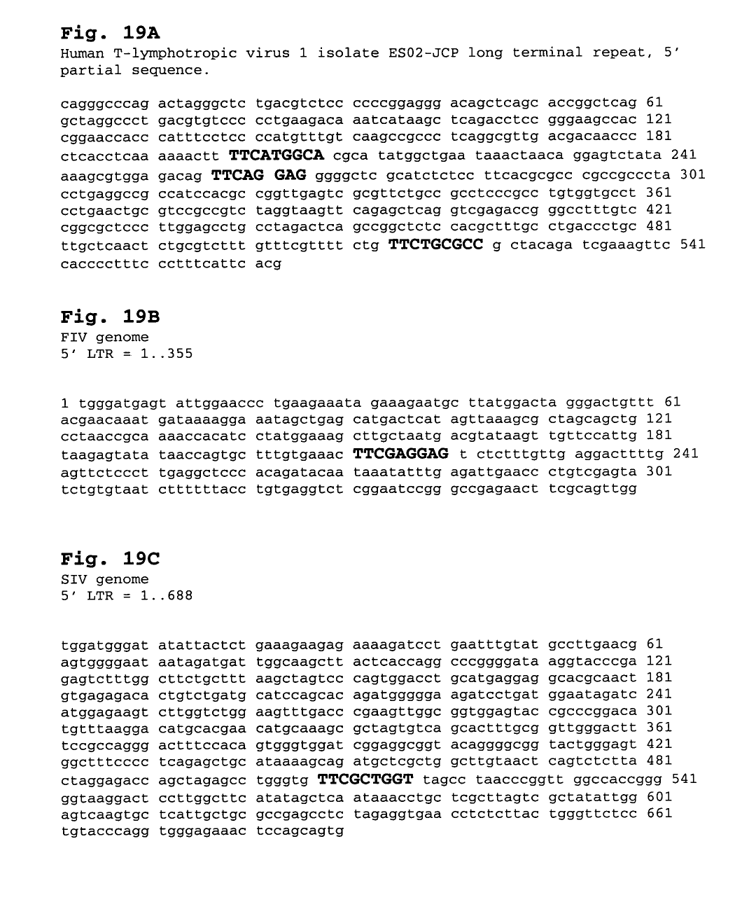

In yet another embodiment of the invention, STAT5 activity may be modulated by any of the methods described hereinabove for JAK3. JAK3 is known to phosphorylate STAT5, among other activities, and certain of the activities associated with JAK3 can be mediated through this phosphorylation of STAT5. Examples of the modulation of STAT5 activity are described in more detail hereinbelow. As indicated in FIG. 19, STAT5 binding site candidates are also present in the 5′ long terminal repeats of the lentivruses human T-lymphotropic virus 1 (HTLV-1), feline immunodeficiency virus (FIV), and simian immunodeficiency virus (SIV). Therefore, the methods of the instant invention may be extended to these viruses for the modulation of the latency of these viruses and for methods to treat cells and patients infected with these viruses.

As used herein, the phrase “STAT5 action” refers to the expression of STAT5 (i.e., transcription and/or translation) and/or any biological activity (i.e., function(s)) exhibited or performed by a naturally occurring form of STAT5 as measured or observed in vivo (i.e., in the natural physiological environment of the protein) or in vitro (i.e., under laboratory conditions). For example, STAT5 action can include, but is not limited to, STAT5 transcription, STAT5 translation, phosphorylation of STAT5, STAT5 protein binding or associating activity (e.g., to a DNA or other STAT proteins), STAT5 protein translocation within a cell, and/or transcriptional regulation of genes by STAT5. An increase in STAT5 action, including an increase in STAT5 expression or an increase in the biological activity of STAT5, can also be referred to as amplification, overproduction, activation, enhancement, up-regulation or increased action of STAT5. An increase in STAT5 action is any measurable increase in STAT5 action in a cell as compared to a control cell in which STAT5 action is intentionally maintained, and/or in which the level of STAT5 action in the control cell is specifically designated to serve as a base-line measurement. Similarly, a decrease in STAT5 action, including a decrease in STAT5 expression or a decrease in the biological activity of STAT5, can also be referred to as inactivation (complete or partial), down-regulation, or reduced or diminished action of STAT5. A decrease in STAT5 action is any measurable decrease in STAT5 action in a cell as compared to a control cell in which STAT5 action is intentionally maintained, and/or in which a level of STAT5 action in the control cell is specifically designated to serve as a base-line measurement.

A modulation in STAT5 action can be measured by any suitable method, including, but not limited to: measurement of STAT5 transcription (i.e., determining STAT5 mRNA levels), measurement of STAT5 translation (determining STAT5 protein levels, e.g., by flow cytometry, immunoblot or other appropriate technique), measurement of phosphorylation of STAT5, measurement of STAT5 protein binding activity (e.g. binding or association with a DNA or other STAT protein), measurement of STAT5 protein translocation within a cell, and/or measurement of transcriptional regulation of genes by STAT5.

As with the JAK3 aspect of the invention, compounds and agents that modulate STAT5 action are suitable for use in the treatment of HIV-infected patient at any stage of disease progression. At least one of the STAT5 action modulating compounds and agents may be delivered to a patient in any pharmaceutically acceptable delivery vehicle, as described hereinabove, such as, without limitation, retroviral vectors, liposomes, protein delivery vehicles, nucleic acid molecules, and the like. Such delivery vehicles may be engineered to specifically target certain cell types such as CD4⁺ T lymphocytes. The administration routes include in vivo and ex vivo routes. Furthermore, these compounds and agents may be delivered in coordination with other agents used to treat HIV infection, as described hereinabove, including agents that modulate JAK3 action.

According to one aspect of the present invention, STAT5 action is increased in CD4⁺ T lymphocytes by administering to the CD4⁺ T lymphocytes of the HIV-infected patient a composition that contains at least one compound that increases the action of STAT5 in the CD4⁺ T lymphocytes. Preferably, the CD4⁺ T lymphocytes include both HIV-infected and uninfected CD4⁺ T lymphocytes that are CD4-ligated.

According to another aspect of the invention, various types of compounds are described that can modulate STAT5 action. Notably, any of the compounds described hereinabove to modulate JAK3 action may modulate STAT5 action. For example, compounds that bind to and stimulate a γ_{c} chain containing T cell surface receptor are suitable in the instant invention. As noted hereinabove, these compounds include, without limitation, certain cytokines, ligands, antibodies, and fusion proteins. Furthermore, compounds that control the transcription of STAT5, such as, without limitation, transcription control factors and homologues thereof that selectively associate with the transcription control sequence of a gene encoding STAT5, are suitable in the present invention.

In another embodiment, a compound suitable for use in the method of the present invention includes a STAT5 protein or another protein to be delivered intracellularly to a suitable host cell, which is operatively linked to an N-terminal protein transduction domain from HIV TAT. As described hereinabove, the HIV TAT construct for use in such a protein is described in detail in Vocero-Akbani et al. (1999) Nature Med., 5:23-33. Such fusion proteins are only cleaved in cells where HIV is present (i.e., HIV-infected cells) by the HIV protease and the active form of the protein attached to TAT is released. The present invention incorporates the use of this technology, “TAT-peptide technology” to deliver proteins for use in the present method (e.g., STAT5 and other proteins which increase the action of STAT5) to cells of a recipient with nearly 100% efficiency, whereby, in one embodiment, the active form of the protein will be released only within the desired target cells. For example, the TAT peptide construct can be engineered so that cleavage of a biologically active form of STAT5 occurs only in T lymphocytes, by the action of, for example, a T cell-specific protease which cleaves gp160 into gp120 and gp41 (described in detail in U.S. Pat. No. 5,691,183, to Franzusoff et al.), or in HIV infected T lymphocytes, using the HIV proteolytic sites for cleavage by an HIV protease, as described in Vocero-Akbani et al.

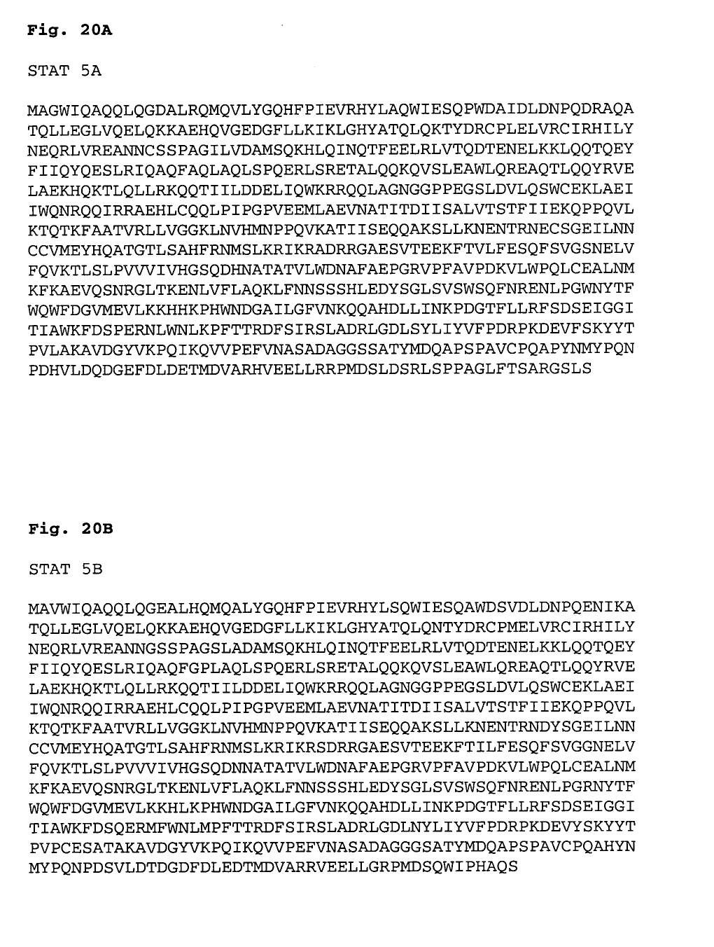

In yet another aspect, a compound suitable for use in the present invention includes recombinant nucleic acid molecules comprising an isolated nucleic acid sequence encoding a biologically active STAT5 protein (see, e.g., GenBank accession numbers NM_{—}003152 and NM_{—}012448; see FIG. 20). As used herein, a biologically active STAT5 includes a full-length STAT5 protein and homologues of STAT5, such as, without limitation, a STAT5 protein wherein amino acids have been deleted, inserted, inverted, substituted, and/or derivatized and wherein the homologue maintains at least one biological function of a naturally occurring, full-length STAT5 protein. As with the JAK3 nucleic acids of the invention, the STAT5 nucleic acids can be produced by any method, such as, obtaining from a natural source and employing recombinant DNA technologies. The isolated nucleic acid sequence may be operably linked to a transcription control sequence to allow for expression of the STAT5 protein when the nucleic acid is present in a desired host cell. In a preferred embodiment of the invention, the isolated nucleic acid is in a vector. A vector, as used herein, is carrier DNA molecule, such as a plasmid, cosmid, bacmid, phage or virus, into which a DNA sequence can be inserted for introduction into a host cell. In another preferred embodiment, the desired target host cells for the nucleic acid of the invention is a CD4-ligated T lymphocyte in an HIV-infected patient. The STAT5 nucleic acid may be introduced to the host by either ex vivo or in vivo methods as described hereinabove for JAK3 nucleic acids.

Nucleic acids of the instant invention may also include antisense nucleic acids and small interfering RNA (siRNA). Antisense nucleic acid molecules may be targeted to translation initiation sites and/or splice sites to inhibit the expression of STAT5 or modulators of STAT5 action. Such antisense molecules are typically between 15 and 30 nucleotides in length and often span the translational start site of mRNA molecules. Antisense constructs may also be generated which contain the entire sequence of STAT5 or modulator of STAT5 action in reverse orientation. siRNA molecules designed to inhibit expression of STAT5 or modulators of STAT5 action are typically double-stranded RNA molecules between about 12 and 30 nucleotides in length, more typically about 21 nucleotides in length. The nucleotide sequence of the siRNA molecules commonly begin from an AA dinucleotide sequence near the AUG start codon but often not within about 75 bases of said start codon. The siRNA molecules typically have a GC content of between about 45% and about 55% and ideally do not contain stretches of more than 3 guanosine bases in a row.

An example of a modulator of STAT5 action that may be down-regulated by an antisense nucleic acid or siRNA in order to increase STAT5 action is HIV Nef (see Example 11). Additionally, compounds that disrupt the ability of Nef to interact with the JAK3/STAT5 pathway or decrease Nef production and methods to screen for such compounds are included in the instant invention.

The methods and compositions of the present invention are suitable for use in any patient with an HIV infection. In particular, the present methods and compositions are suitable for use in any HIV-infected patient in which there is a reasonable likelihood that a therapeutic benefit can be obtained by the use of such method or composition. Such a patient can be characterized as having a sufficient number of “STAT5 rescueable CD4⁺ T cells” such that increasing immune responsiveness in these T lymphocytes by the method or composition of the present invention would be reasonably expected to provide a measurable benefit to the patient, alone or in combination with other HIV therapies. As used herein, a “STAT5 rescueable T lymphocyte” is a T lymphocyte with reduced immune responsiveness in which STAT5 action can be increased by the method or composition of the present invention, such increase being sufficient to increase immune responsiveness in the T lymphocyte.

Another embodiment of the present invention relates to a method to identify a regulatory compound that modulates immune responsiveness in an HIV-infected CD4⁺ T lymphocyte by modulating STAT5 action. The method includes the steps of: (a) contacting a resting CD4⁺ T lymphocyte with a CD4-ligating compound that selectively binds to CD4 on said CD4⁺ T lymphocyte; (b) contacting the CD4⁺ T lymphocyte, after step (a), with a stimulatory compound that stimulates T cell receptor-mediated activation of the CD4⁺ T lymphocyte; (c) contacting the CD4⁺ T lymphocyte with a putative regulatory compound; and, (d) determining whether STAT5 action is modulated in said CD4⁺ T lymphocyte. A modulation in STAT5 action in the test CD4⁺ T lymphocyte, as compared to STAT5 action in a control CD4⁺ T lymphocyte that has not been contacted with the putative regulatory compound, indicates that the putative regulatory compound modulates immune responsiveness in CD4⁺ T lymphocytes from an HIV-infected patient. A similar method has been discussed in detail hereinabove for JAK3 action modulation.

As noted hereinabove, step (a) can be achieved by a variety of methods such as contacting cells with a CD4-ligating compound and isolating HIV-infected T lymphocytes from an HIV-infected patient. Furthermore, the nature of the stimulatory compounds employed in step (b) are detailed hereinabove. Step (c), in addition to be performed after step (b) and before step (d), may also performed after step (a) but before step (b) or before step (a) to address different mechanisms of the modulation of STAT5 action as described hereinabove for the modulation of JAK3 action. The putative regulatory compounds can be obtained, for example, from molecular diversity strategies, libraries of natural or synthetic compounds, or by rational drug design as described above.

Yet another embodiment of the present invention relates to a method to increase CD4⁺ T lymphocyte immune responsiveness in a patient having human immunodeficiency virus (HIV) infection. The method includes the step of administering to the patient a composition which includes: (a) a compound that selectively binds to and stimulates a receptor having a γ_{c} chain on the surface of CD4⁺ T lymphocytes in the patient, wherein the compound is administered in an amount sufficient to increase STAT5 action in the CD4⁺ T lymphocytes; and, (b) a pharmaceutically acceptable delivery vehicle that specifically targets the CD4⁺ T lymphocytes. In one embodiment, the patient to which such a composition is administered is characterized as having a CD4⁺ T cell count of at least about 100 cell/mm³ and an HIV viral titer of less than about 400 copies/ml as determined by plasma RNA PCT within 30 days of when the method is employed. Details of such a method have been previously described in detail herein.

Yet another embodiment of the present invention relates to a method to increase CD4⁺ T lymphocyte immune responsiveness in a patient having human immunodeficiency virus (HIV) infection. Such method includes the steps of administering to the patient a composition which includes: (a) a compound selected from the group of: (1) a cytokine selected from the group of interleukin-7 (IL-7), IL-9, IL-13 and/or IL 15; (2) an antibody that selectively binds to a receptor comprising a γ_{c} chain; (3) a compound that increases the expression of STAT5 in the CD4⁺ T lymphocytes by associating with a transcription control sequence of a gene encoding STAT5 such that STAT5 transcription is increased; (4) a STAT5 protein or biologically active fragment thereof, operatively linked to an N-terminal protein transduction domain from HIV TAT; and/or, (5) a recombinant nucleic acid molecule comprising an isolated nucleic acid sequence encoding a biologically active STAT5 protein operatively linked to a transcription control sequence. The compound is administered in an amount sufficient to increase STAT5 action in the CD4⁺ T lymphocytes. The composition additionally includes (b) one or more anti-retroviral therapeutic compounds. In one embodiment, the patient to which such a composition is administered is characterized as having a CD4⁺ T cell count of at least about 100 cells/mm³ and an HIV viral load of less than about 400 copies/ml when the method is employed. Details of such a method have been previously described in detail herein.

Another embodiment of the present invention relates to a method to identify an HIV-infected patient as a suitable candidate for employment of a method to increase CD4⁺ T lymphocyte responsiveness as previously described herein. The method includes the steps of: (a) isolating a sample of T lymphocytes from the patient; (b) stimulating the T lymphocytes with a stimulatory compound that stimulates T cell receptor-mediated activation of the T lymphocytes, the step of stimulating being performed in the presence and absence of a compound that binds to and activates a cytokine receptor having an γ_{c} chain; (c) measuring STAT5 action in the T lymphocytes of step (b) and, (d) identifying candidate patients in which the sample of T lymphocytes shows a measurable increase of at least about 10% in STAT5 action in the presence of the compound as compared to in the absence of the compound.

Yet another embodiment of the present invention relates to methods to identify compounds which modulate the amount of latently HIV-infected T cells in an HIV-infected patient. Such methods can include an in vivo method and in vitro assay. Notably, compounds determined to reduce the amount of latently infected cells can be effective in preventing the formation of latently infected cells. The in vivo method includes the steps of (a) isolating a first sample of T lymphocytes from an HIV-infected patient; (b) measuring an amount of latently infected T lymphocytes in the first sample; (c) administering to the patient in vivo a composition comprising one or more compounds that increase the action of STAT5 in the CD4⁺ T lymphocytes; d) isolating a second sample of T lymphocytes from the HIV-infected patient, after step (c); (e) measuring an amount of latently infected T lymphocytes in the second sample. A decrease in the amount of latently infected CD4⁺ T lymphocytes in the second sample as compared to the amount of latently infected CD4⁺ T lymphocytes in the first sample indicates that the composition is effective to eliminate latently infected CD4⁺ T lymphocytes in the patient. Methods of isolating and measuring amounts of latently HIV-infected T cells are known in the art and have been previously described herein (see, e.g., Chun et al. (1997) Nature 387:183-188; Kutsch, O., et al. (2002) J. Virol., 76:8776-86).

The in vitro method includes the steps of (a) isolating a sample of T lymphocytes from an HIV-infected patient; (b) measuring an amount of latently infected T lymphocytes in the first sample; (c) contacting the lymphocytes with a panel of compounds that bind to and activate a cytokine receptor having an γ_{c} chain; (d) identifying a compound from the panel of compounds wherein the T lymphocytes show a larger increase in STAT5 action in the presence of the compound as compared to in the presence of the other compounds in the panel and/or a larger increase in productive HIV-infection in the presence of the compound as compared to in the presence of the other compounds in the panel; and, (e) selecting and administering the compound showing the larger increase to the patient for elimination of latently HIV-infected CD4⁺ T lymphocytes in the patient.

In yet another embodiment of the invention, methods are provided for the modulation of latency of an HIV-infected cell, preferably a CD4⁺ T lymphocyte. Such a method includes the steps of (a) obtaining HIV-infected cells and (b) contacting the HIV-infected cells with a compound that modulates STAT5 action. The HIV-infected cells of step (a) may be, without limitation, an HIV-infected tissue culture cell line (e.g., WE17/10 cells), in vivo CD4⁺ T lymphocytes, isolated CD4⁺ T lymphocytes infected in vitro, and CD4⁺ T lymphocytes isolated from an HIV-infected patient. Compounds, including nucleic acids, proteins, peptides, chemical compounds, and the like, that modulate STAT5 action are described hereinabove and include compounds that increase STAT5 action and thereby promote transcription of HIV genes and compounds that decrease STAT5 action and thereby promote latency in the HIV-infected cell. The latency status and the state of STAT5 (e.g., activation, phosphorylation) can be monitored thoroughout the experiment.

Yet another embodiment of the present invention relates to a method to identify compounds which modulate latency in HIV-infected cells. Such a method can include an in vivo method and in vitro assay. The in vivo method includes the steps of (a) isolating a first sample of T lymphocytes from an HIV-infected patient; (b) measuring the amount of latently infected T lymphocytes and/or the amount of T lymphocytes actively producing virus in the first sample; (c) administering to the patient in vivo a composition comprising one or more compounds that modulate the action of STAT5 in the CD4⁺ T lymphocytes; (d) isolating a second sample of T lymphocytes from the HIV-infected patient, after step (c); and (e) measuring the amount of latently infected T lymphocytes and/or the amount of T lymphocytes actively producing virus in the second sample. A decrease in the amount of latently infected CD4⁺ T lymphocytes in the second sample as compared to the amount of latently infected CD4⁺ T lymphocytes in the first sample indicates that the composition is effective to eliminate or reduce the number of latently infected CD4⁺ T lymphocytes in the patient. A decrease in the amount of CD4⁺ T lymphocytes actively producing virus (i.e., not latent) and/or an increase in the amount of latently infected cells in the second sample as compared to the amount of CD4⁺ T lymphocytes actively producing virus and latently infected cells in the first sample indicates that the composition is effective to promote latency in HIV-infected CD4⁺ T lymphocytes in the patient. Methods of isolating and measuring amounts of latently HIV-infected T cells and the amount of T lymphocytes actively producing virus in the sample have been previously described herein.

The in vitro method includes the steps of (a) isolating a sample of T lymphocytes from an HIV-infected patient; (b) measuring the amount of latently infected T lymphocytes and/or the amount of T lymphocytes actively producing virus in the sample; (c) contacting the lymphocytes with a composition comprising one or more compounds that modulate the action of STAT5 in the CD4⁺ T lymphocytes; and (d) measuring the amount of latently infected T lymphocytes and/or the amount of T lymphocytes actively producing virus in the sample. A decrease in the amount of latently infected CD4⁺ T lymphocytes in the sample after contact with the modulating compound as compared to the amount of latently infected CD4⁺ T lymphocytes in the sample prior to contact with the modulating compound indicates that the composition is effective to activate HIV from the latent state in infected CD4⁺ T lymphocytes. A decrease in the amount of CD4⁺ T lymphocytes actively producing virus (i.e., not latent) and/or an increase in the amount of latently infected cells in the second sample as compared to the amount of CD4⁺ T lymphocytes actively producing virus and amount of latently infected cells in the first sample indicates that the composition is effective to promote latency in HIV-infected CD4⁺ T lymphocytes. Compounds identified in this in vitro method may be administered to patients to modulate latency in vivo.

Alternatively, the in vitro method may be performed on HIV latent cell models. Such HIV latent cell models include, without limitation, JNL-GFP which is a Jurkat T cell line latently infected with NL4-3-GFP (Kutsch, O., et al. (2002) J. Virol., 76:8776-86); and WENL-GFP which is a WE17/10 T cell line latently infected with NL4-3-GFP obtained by the method of Kutsch et al. GFP expression can be directly monitored by flow cytometry and the like and STAT5 activation and phosphorylation and JAK3 and Nef expression may also be readily assessed as described hereinabove.

In another embodiment of the invention, methods are provided for treating a patient who has been infected by a microbe that is associated with latent infections. The methods of the invention comprise the administration of at least one of the group consisting of: pentostam (stibogluconate sodium), rapamycin, and derivatives or analogs thereof.

Rapamycin is in common clinical use and has been approved by the FDA for use as an immunosuppressive agent after solid organ transplant (Ettenger & Grimm (2001) Am. J. Kidney Dis. 38:S22-28; reviewed in Saunders et al. (2001) Kidney Int. 59:3-16). Rapamycin has recently been shown to inhibit HIV-1 replication in human T cells in vitro (Roy et al. (2002) Antimicrob. Agents Chemo. 46:3447-3455). Additionally, rapamycin has been shown to inhibit HIF-1, which is a transcriptional activator of HALP/RTP801, through inhibition of mTOR, mammalian target of rapamycin (Hudson et al. (2002) Mol. Cell. Biol. 22:7004-7014; Shoshani et al. (2002) Mol. Cell. Biol. 22:2283-2293). Notably, HALP blocks apoptosis and maintains survival of HIV-infected cells (U.S. patent application Ser. No. 10/368,803; Selliah et al. (2003) Immunol. Res. 27:247-260; Yin et al. (2004) AIDS 18:587-596). Methods for preparing rapamycin, its derivatives and analogs, and pharmaceutical compositions comprising the same are well know in the art and have been disclosed in the following exemplary list of U.S. Pat. Nos. 4,316,885; 4,650,803; 5,100,883; 5,117,203; 5,118,677; 5,118,678; 5,130,307; 5,194,447; 5,258,389; 5,262,423; 5,665,772; 5,985,325; 6,004,973; 6,197,781; 6,200,985; 6,277,983; 6,342,507; 6,503,883; 6,599,535 and 6,710,053; the entire contents of which are incorporated herein by reference. In a preferred embodiment of the instant invention, derivatives and analogs of rapamycin to be employed in the instant invention possess the ability to inhibit mTOR. In a more preferred embodiment, the derivatives and analogs of rapamycin to be employed in the instant invention possess the ability to inhibit HIF-1.

Pentostam (stibogluconate sodium) has been previously employed in clinical use for treating leishmaniasis in both HIV-infected and uninfected hosts. Notably, pentostam has been shown to inhibit cellular protein tyrosine phosphatase (PTP), particularly PTP1B, thereby promoting the activity and phosphorylation of STAT5 (Pathak & Yi (2001) J. Immunol. 167:3391-3397). Derivatives and analogs of Pentostam include, for example, pentavalent antimony compounds (e.g., potassium hexahydroxy antimonate; meglumine antimoniate (Glucantime; Rhone-Poulenc, Paris, France)) and trivalent antimony compounds (e.g., as potassium antimony tartrate). Preferably, derivatives and analogs of pentostam to be employed in the instant invention possess the ability to inhibit PTP1B. More preferably, the derivatives and analogs of pentostam to be employed in the instant invention possess the ability to increase the activity of STAT5.

The administration of at least one of the group consisting of: pentostam (stibogluconate sodium), rapamycin, and derivatives or analogs thereof, to a patient in need thereof, results in the reduction, elimination, and/or prevention of the formation of a latent reservoir. In a preferred embodiment, both pentostam or a derivative or analog thereof is administered in combination with rapamycin or a derivative or analog thereof. In a particular embodiment, the agents may be administered at the same time or at different times. In a particular embodiment, the present method is used in a patient during an acute infection by the microbe. In yet another embodiment, the patient is undergoing a structured treatment interruption (STI).

Pentostam, rapamycin, and derivatives or analogs thereof can be administered as a composition in a pharmaceutically acceptable delivery vehicle, described hereinabove. The compositions may also be administered by any route described hereinabove. Additionally, targeting delivery vehicles as described hereinabove may also be employed. Exemplary administration methods, dosing amounts, and pharmaceutically acceptable delivery vehicles for penstostam are provided by GlaxoSmithKline (Research Triangle Park, N.C.; see, e.g., emc.medicines.org.uk/emc/assets/c/html/displaydoc.asp?documentid=2182). Exemplary pharmaceutically acceptable delivery vehicles for oral administration of rapamycin are provided, for example, in U.S. Pat. Nos. 5,985,325; 5,536,729 and 5,559,121. Intravenous rapamycin formulations are disclosed, for example, in U.S. Pat. Nos. 5,516,770 and 5,530,006. Effective dose parameters for the instant invention may vary from those determined for pentostam and rapamycin in other uses (e.g., treatment of leishmania). Thus, effective dose parameters for the instant invention may be determined using methods standard in the art. Such methods include, for example, determination of survival rates, side effects (i.e., toxicity), determination of cellular immune response effects, and progression or non-progression of microbe-related conditions. In particular, the effectiveness of dose parameters of a composition of the present invention when treating infection by microbes which cause latent infections can be determined by assessing response rates. Such response rates refer to the percentage of treated patients in a population of patients that respond with measurable improvement in preventing or reducing the number of cells latently infected by the microbe.

In accordance with the present invention, the latent reservoir of the microbe in the patient can be monitored by measuring at least one of the group consisting of: microbe RNA (e.g., plasma RNA), microbe DNA (e.g., from host cells), CD4 cell levels, infectious microbes by co-culture with other cells (e.g., PBMCs), p24 expression for HIV, and IFN-γ secretion by CD8 cells. Such methods are known to those skilled in the art (see, for example, Ausubel et al., eds. (1998) Current Protocols in Molecular Biology, John Wiley and Sons, Inc.).

In yet another embodiment of the instant invention, other anti-microbial agents (e.g., anti-viral agents, anti-parasitic agents, anti-bacterial agents) can be co-administered with the compositions of the instant invention. In a particular embodiment of the invention, the host is, subsequent to treatment with compositions of the instant invention, treated with highly active antiretroviral therapy (HAART), particularly when the microbe is HIV-1.

The following examples illustrate various aspects of the present invention. They are not to be construed to limit the claims in any manner whatsoever.

### EXAMPLE 1

The following example shows that stimulation through γ_{c}-related cytokine receptors rescues T cells from gp120 or anti-CD4 mediated inhibition of T cell activation.

Heparinized venous blood from healthy adult human donors was separated on a Ficoll-Paque (Pharmacia Biotech) gradient to obtain lymphocytes. CD4⁺ T cells were isolated by incubation with anti-CD8 mAb (OKT8, 20 Ag/ml, ATCC), followed by negative selection on goat anti-mouse IgG coated Immulan beads (Biotecx Laboratories). Isolated cells were determined to be 80-95% CD4+ by flow cytometric analysis (data not shown).

To determine whether cytokines would reverse gp120- or anti-CD4-mediated T cell unresponsiveness, the purified human CD4⁺ T cells were incubated with HIV surface glycoprotein, gp120, or Leu-3a (an antibody that binds to the gp120 binding site on CD4) in the presence or absence of either IL-2, IL-4, IL-7, IL-6 or IL-12. Specifically, the purified CD4⁺ T cells in balanced salts solution were incubated with or without gp120 (rgp120SF2, 10 μg/ml) and crosslinked with anti-gp120 antibody (1:250 dilution) or anti-CD4 mAb (anti-CD4, Leu-3α, 20 μg/ml) for 1 hr at 37° C.

Proliferation in response to plate-bound anti-TCR was determined as follows. Cells were washed, resuspended in RPMI-1640 culture media (GIBCO) supplemented with 10% fetal bovine serum (FBS, Gemini BioProducts) and 1×10⁵ cells were added to triplicate wells of an anti-T cell receptor monoclonal antibody-coated (anti-TCR, BMA-031, 50 μg/ml) 96-well plate (Becton Dickinson). 20 U/ml IL-2, IL-4, IL-6, IL-12 (R&D Systems) or IL-7 (Genzyme) were added to the culture. Culture plates were incubated for 3 days at 37° C. with 1 μCi/Well [³H]-thymidine (NEN) present during the final 5 hours of culture. The cells were harvested and processed to determine ³H-thymidine incorporation.

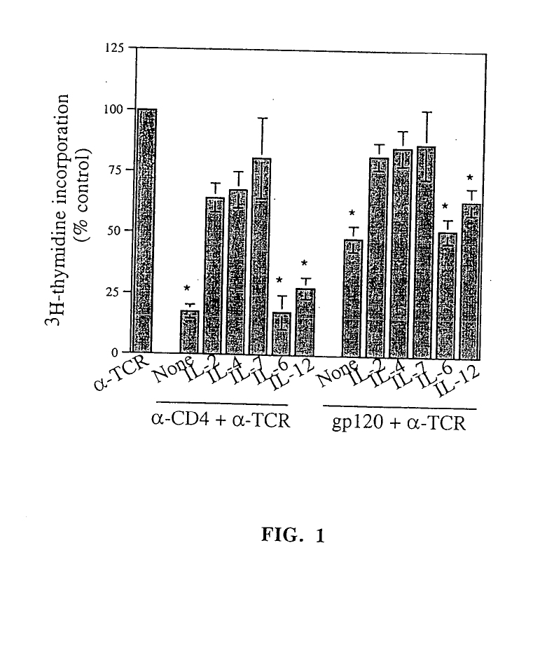

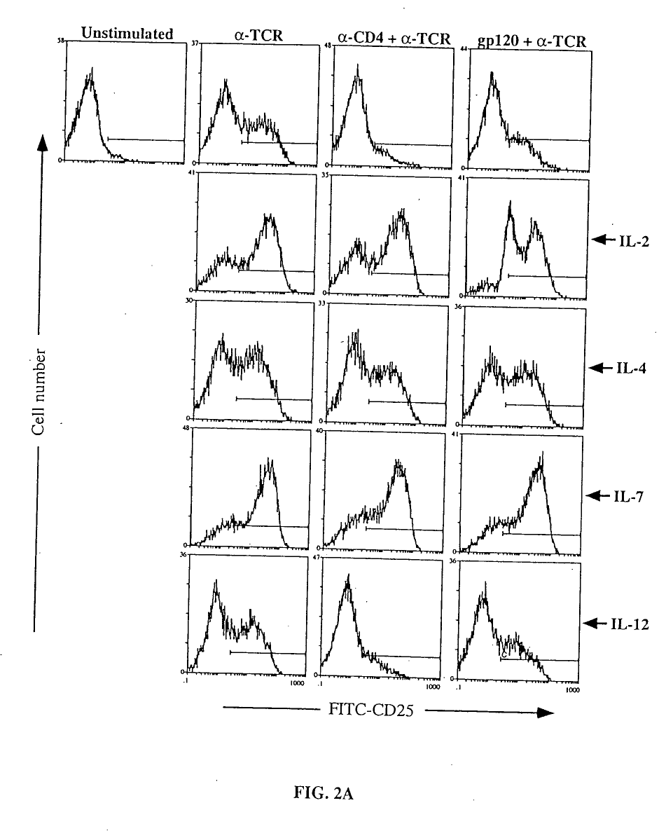

As shown in FIG. 1, ligation of CD4 prior to T cell activation through the T cell receptor inhibited anti-TCR induced proliferation. Addition of exogenous IL-2. IL-4 or IL-7 (cytokines which bind to receptors on T cells having γ_{c}), but not IL-6 or IL-12 (cytokines that bind to receptors on T cells which lack γ_{c}), restored the proliferative response. Higher concentrations of IL-6 or IL-12 (up to 80 U/ml) did not reverse the inhibition of proliferation (data not shown).

### EXAMPLE 2

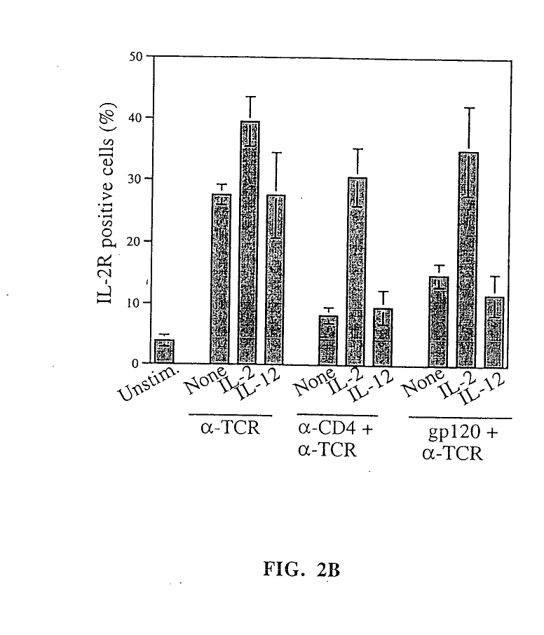

The following example demonstrates that addition of cytokines which bind to receptors having γ_{c}, restores activation induced CD25 expression on CD4 primed T cells. Expression of high affinity IL-2R (CD25) was also analyzed in CD4 primed T cells. To determine the expression of IL-2R (α-chain, CD25), the culture plates were set up as described in Example 1 and incubated at 37° C. for 24 hours. 2×10⁵ cells were stained with FITC-conjugated anti-CD25 mAb (Pharmingen) and analyzed by flow cytometry (Coulter XL). As shown in FIGS. 2A and 2B, addition of exogenous IL-2, IL-4 or IL-7, but not IL-6 (data not shown) or IL-12, restored activation-induced CD25 expression. These data show that ligation of CD4 by HIV gp120 inhibits T cell activation, and that T cell function can be restored by engagement of cytokine receptors that share the common γ_{c} chain.

### EXAMPLE 3

The following example shows that gp120 or anti-CD4 inhibits-T cell receptor-induced expression and activation of JAK3.

In this experiment, the present inventors determined the activation status of JAK3 in CD4⁺ T cells which were activated through the TCR subsequent to CD4 ligation. CD4⁺ T cells were isolated and stimulated through TCR/CD3 with or without prior CD4 ligation as described in Example 1, and activation of JAK3 was determined.

Briefly, the purified CD4⁺ T cells were incubated with or without gp120 and anti-gp120 antibody or anti-CD4 for 1 hour at 37° C. 3×10⁶ cells per well were incubated at 37° C. in an anti-CD3 mAb coated (anti-CD3, OKT3, 50 μg/ml, ATCC) 12-well plate. Cells were harvested after various times and lysed in Tris-buffered saline (TBS) containing 1% NP-40, phosphatase inhibitors and protease inhibitors. After micro centrifugation, the post-nuclear lysates were used for immunoprecipitation with anti-JAK3 polyclonal antibody (anti-JAK3, 10 μl, Santa Cruz Biotechnology). The antibody-protein complex was pelleted using Sepharose-conjugated Protein A (Sigma) boiled in sample buffer (0.4% SDS, 3% glycerol and 1% β-mercaptoethanol (2-ME)) and the proteins were separated by 7.5% SDS-PAGE. The proteins were transferred to nitrocellulose membrane and immunoblotted with antiphosphotyrosine mAb (anti-P-Tyr, Ab-2, Oncogene Science) Positive protein bands were detected with horseradish peroxidase (HRP)-conjugated goat anti-mouse IgG (Jackson ImmunoResearch) and SuperSignal Substrate (SSS, Pierce). Membranes were stripped with 62.5 mM Tris-HCl, pH 6.7, containing 10 mM 2-ME, and 2% SDS, immunoblotted with anti-JAK3, and developed with HRP-conjugated protein A and SSS.

In some experiments, 1.5×10⁶ cell equivalents of post-nuclear lysate were boiled with sample buffer and separated by 7.5% SDS-PAGE. Nitrocellulose membrane was immunoblotted with (anti-JAK3 and then stripped and immunoblotted with anti-actin mAb (anti-actin, Sigma), as described above. Optical density (O.D.) of positive bands was measured with the Stratagene Eagle Eye II.

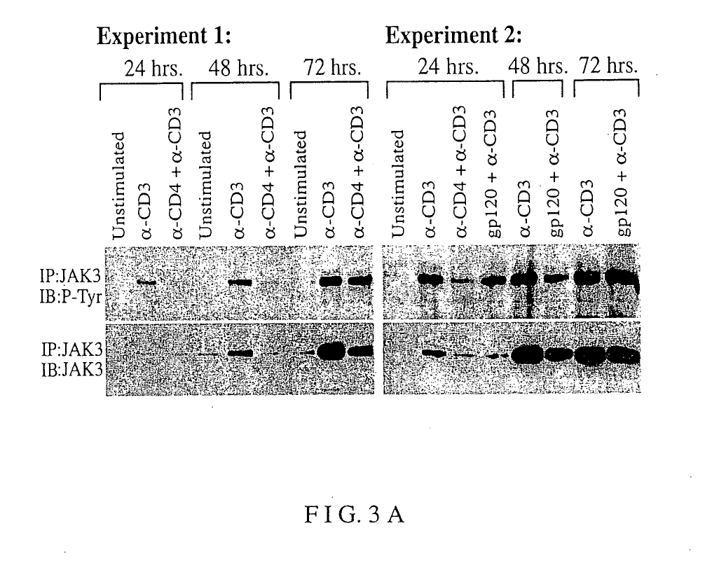

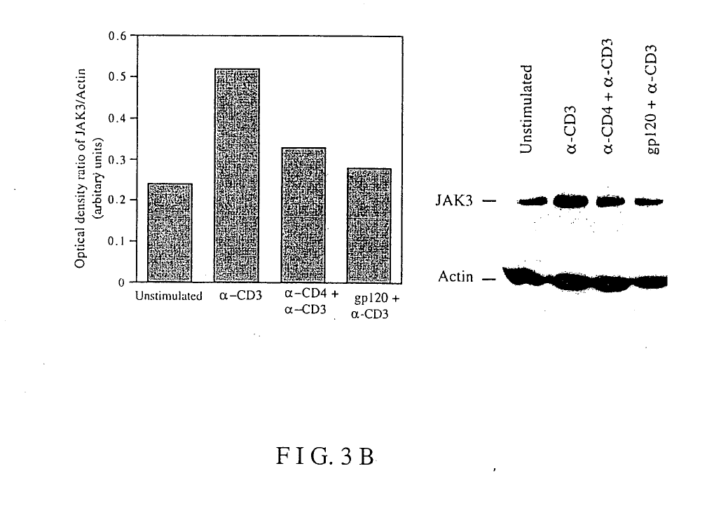

Activation of JAK3 is accompanied by autophosphorylation of tyrosine residues (Johnston et al. (1994) Nature, 370:151) FIG. 3A shows that very low levels of JAK3 protein were expressed in resting T cells, and that stimulation through TCR/CD3 increased the expression and tyrosine phosphorylation of JAK3 in a temporal manner. Surprisingly, prior CD4 ligation with gp120 or anti-CD4 inhibited the TCR/CD3-induced expression as well as the phosphorylation of JAK3 (FIG. 3A). This result was confirmed by Western blotting of whole cell lysates with anti-JAK3 and anti-actin. As shown in FIG. 3B, resting T cells expressed low levels of JAK3, and TCR/CD3 stimulation induced increased JAK3 expression, when normalized to the actin control. Prior CD4 ligation with gp120 or anti-CD4, however, inhibited the TCR/CD3 induced expression of JAK3 (FIG. 3B). These data show that gp120 or anti-CD4 mediated T cell unresponsiveness is correlated with inhibition of JAK3 expression and activation.

As shown in FIG. 3A, the inhibition of JAK3 expression and activation was essentially complete in response to CD4 ligation with anti-CD4, but incomplete in response to ligation with gp120. Although the reasons for this difference between gp120 and anti-CD4 are unclear, without being bound by theory, the present inventors believe that coligation of CD4 and the chemokine receptor, CXCR4, by gp120 (D'Souza and Harden (1996) Nature Med., 2:1293) may be playing a role in differential signaling. In addition, while JAK3 was significantly inhibited in CD4 primed cells after 24 and 48 hrs of stimulation, expression and activation of JAK3 were noted after 72 hrs. This was correlated with an increase in IL-2R expression (data not shown), although these cells did not proliferate in response to anti-TCR (Example 1, FIG. 1), and addition of IL-2 after 24 hrs of activation failed to rescue CD4 primed T cells (data not shown). These data suggest that an early window of opportunity exists for rescue of T cell function by γ_{c} cytokines.

### EXAMPLE 4

The following example demonstrates that activation of JAK3, but not JAK1, correlates with rescue of CD4 mediated T cell unresponsiveness.

As shown in Examples 1 and 2, engagement of γ_{c}-related cytokine receptors restored CD4 ligation-mediated inhibition of T cell activation. Therefore, the activation status of JAK3 in these rescued cells was determined. CD4⁺ T cells were isolated and stimulated through TCR/CD3 with or without prior CD4 ligation as described in Example 1, 20 U/ml IL-2, IL-7 or IL-12 were added to the cultures, and activation of JAK3 was determined as described in Example 3.

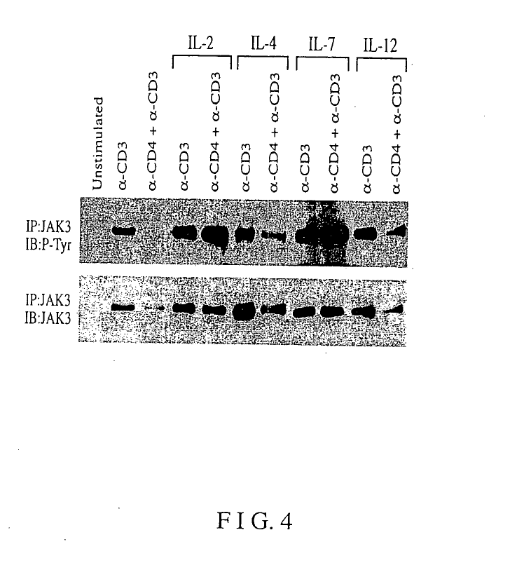

Addition of exogenous IL-2, IL-4 (data not shown) or IL-7, but not IL-12, completely reversed the gp120 (data not shown) or anti-CD4 induced inhibition of JAK3 expression and activation (FIG. 4). These data show that rescue of CD4 ligation-mediated inhibition of T cell activation correlates with activation of JAK3.

Another Janus family kinase, JAK1, associates with the β chain of IL-2R and with the α chains of IL-4R and IL-7R, and is autophosphorylated upon activation (Johnston et al. (1994) Nature, 370:151; and Russell et al. (1994) Science, 270:797). The activation of JAK1 was analyzed in T cells stimulated through TCR/CD3 with or without prior CD4 ligation with anti-JAK1 polyclonal antibody (anti-JAK1, 10 μl, Santa Cruz Biotechnology) as described for JAK3 in Example 3.

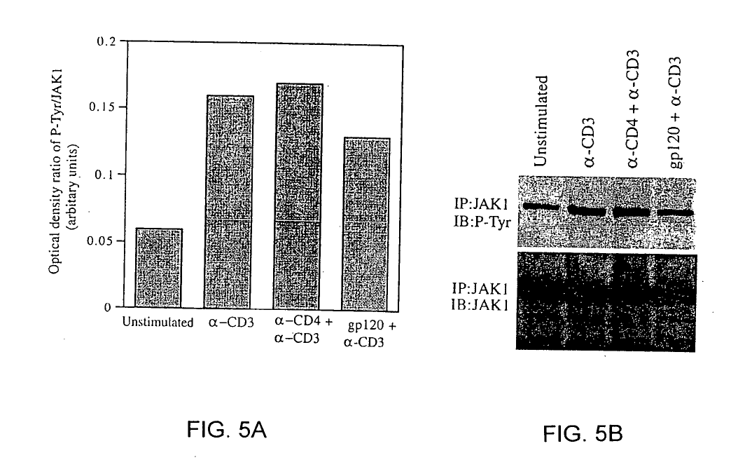

As shown in FIG. 5, JAK1 is expressed constitutively, and a low level of phosphorylation is seen in resting T cells. Stimulation through TCR/CD3 increased the phosphorylation of JAK1. However, prior CD4 ligation with gp120 or anti-CD4 did not significantly change the activation status of JAK1 (FIG. 5) Collectively, these data suggest that activation of JAK3, and not JAK1, plays a role in cytokine rescue of CD4 ligation mediated T cell unresponsiveness.

### EXAMPLE 5

The following example demonstrates that CD4⁺ T cell PHA blasts infected in vitro with HIV-1 show inhibition of JAK3 expression upon activation of the T cell.

Purified human CD4⁺ T cells isolated as described above and activated with PHA were infected for 4 days with a laboratory cloned strain of HIV-1, NL4-3. Cultures were supplemented with 20 U/ml recombinant IL-2. After the infection period, 3×10⁶ cells per well were incubated at 37° C. in an anti-CD3 mAb coated (anti-CD3, OKT3, 50 μg/ml) plate for 20 hours or 40 hours without the addition of exogenous IL-2. Cells were harvested at the designated times and Western blots using anti-JAYO or anti-actin were prepared as described in Example 3.

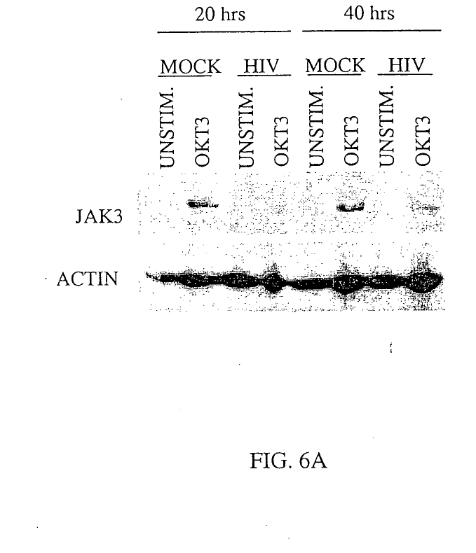

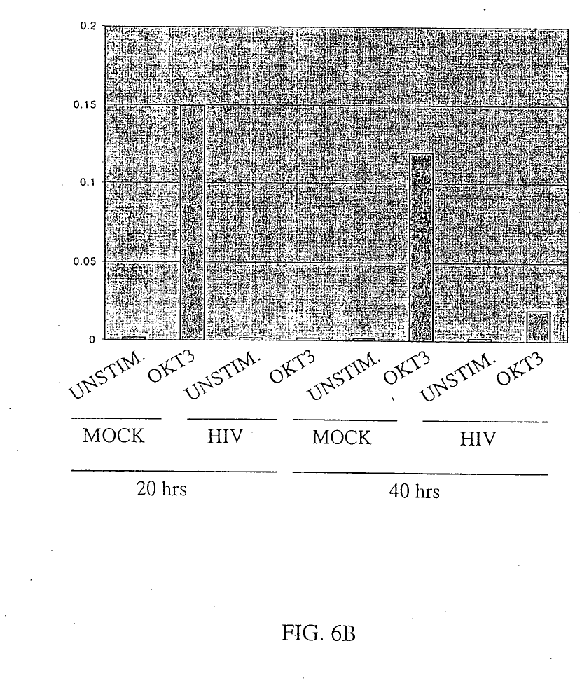

FIGS. 6A and 6B show that at both 20 hours and 40 hours after stimulation with anti-CD3, mock-infected, control CD4⁺ T cells show significantly increased expression of JAK3. In contrast, HIV-infected CD4⁺ T cells showed a marked inhibition of JAK3 expression after stimulation with anti-CD3.

### EXAMPLE 6

The following example demonstrates that T cells isolated from HIV-infected patients show inhibition of JAK3 expression upon activation of the T cell.

In this experiment, peripheral blood T cells were isolated from the venous blood of HIV-positive children donors who had a CD4⁺ T cell count of greater than 500 cells/mm³. Peripheral blood T cells isolated from a healthy, non-infected children donors served as a control. 0.5×10⁶ cells per well (24 well culture plate) were incubated at 37 C in an anti-CD3 mAb coated (anti-CD3, OKT3, 10 μg/ml) plate for 20 hours or 40 hours without the addition of exogenous IL-2. Cells were harvested at the designated times and Western blots using anti-JAK3 or anti-actin were prepared as described in Example 3.

The combined results from the experiments are shown in FIG. 7 as an average O.D. ratio of JAK3/Actin +/− standard deviation. FIG. 7 shows that after stimulation with anti-CD3, T cells from the normal control donors (normal) showed a significant increase in JAK3 expression as compared to unstimulated cells from the same donors. In contrast, the T cells isolated from HIV-infected patients had a much lower initial level of JAK3 expression than the normal controls and JAK3 expression was stimulated to a significantly lesser level by anti-CD3.

### EXAMPLE 7

The following example demonstrates that T cells infected with HIV showed inhibition of JAK3 expression upon activation of the T cell, and are rescued by administration of IL-7.

In this experiment, purified human CD4⁺ T cells isolated as described above and activated with PHA for 3 days were infected for 4 days with a laboratory cloned strain of HIV-1, NL4-3. As a control, a sample of the PHA blasts was infected with a mock virus, which is a retroviral control vector that does not contain HIV. After the infection period, 0.5×10⁶ cells per well were incubated at 37° C. in an anti-CD3 mAb coated (anti-CD3, OKT3, 10 μg/ml) plate for 48 hours in the presence of 20 U/ml recombinant IL-2 or IL-7. Cells were harvested and lysed with TBS/1% NP40 and analyzed on 7.5% SDS PAGE. Western blots using anti-JAK3 or anti-actin were prepared as described in Example 3.

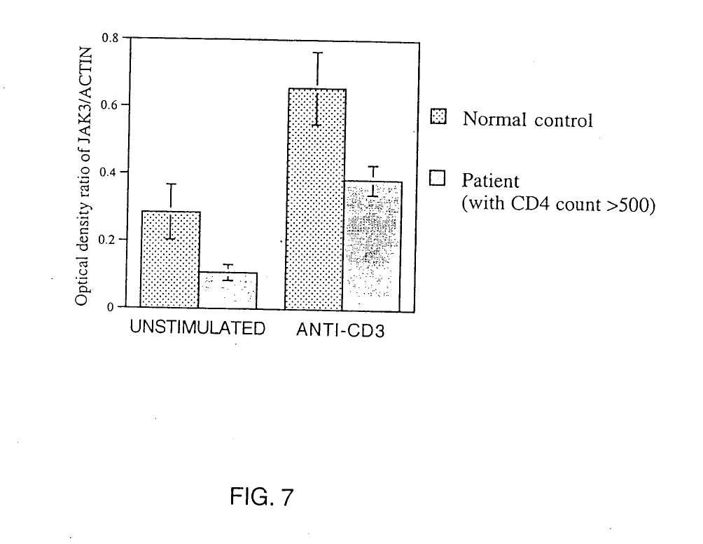

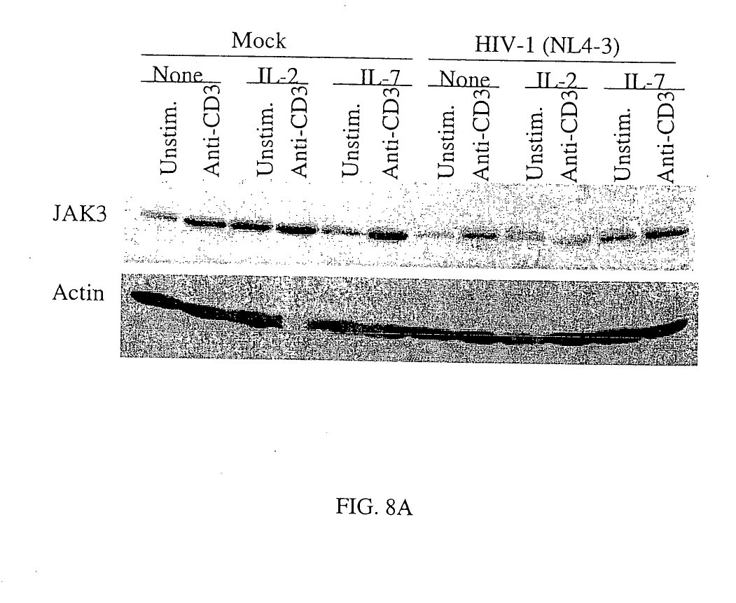

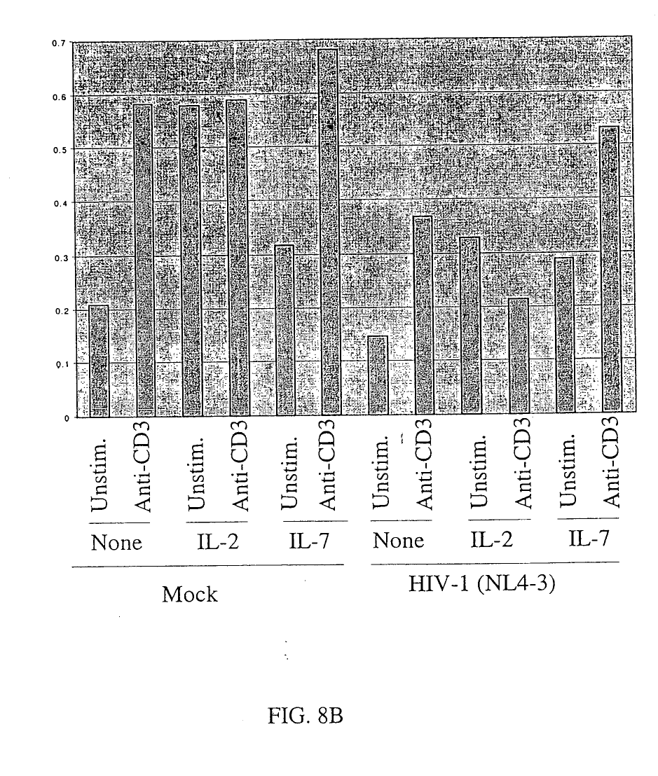

FIGS. 8A and 8B show that in the mock-infected cells (control), cells stimulated with anti-CD3 showed an increase in JAK3 expression as compared to unstimulated cells. The HIV-infected T cells show a lower initial level of JAK3 expression and significantly lower increase in JAK3 expression upon anti-CD3 stimulation.

The addition of IL-2 to the mock-infected and HIV infected cultures resulted in a significant increase in JAK3 expression in the unstimulated T cells. This is likely due to the upregulation of IL-2R on the T cells upon PHA activation (necessary for virus infection of the cells). Therefore, IL-2 in these cells increases the JAK3 expression and induces T cell proliferation, even in the absence of stimulation by anti-CD3.

The addition of IL-7 to the mock-infected T cells increased JAK3 expression slightly in stimulated cells as compared to stimulated cells in the absence of IL-7. In HIV infected T cells, the addition of IL-7 significantly increased JAK3 expression in stimulated cells as compared to stimulated, HIV-infected cells in the absence of IL-7, indicating a positive effect of IL-7 on the immune responsiveness of HIV infected T cells.

### EXAMPLE 8

The following example demonstrates that HIV-1 infection of T lymphocytes inhibits the activation of JAK3 and the kinase activity of JAK3.

In this experiment, purified human CD4⁺ T cells isolated as described above were activated for 3 days with PHA and infected with Mock or HIV-1 (NL4-3) as described above. After 4 days, 5×10⁶ cells were lysed with Tris buffered saline (TBS) containing 1% NP40. Whole cell lysates were immunoprecipitated (IP) with anti-JAK3 antibody and then with anti-STAT5 antibody. Proteins were separated on 7.5% SDS-PAGE and immunoblotted (IB) with anti-phosphotyrosine antibody (P-Tyr). Then, the membranes were stripped and immunoblotted with anti-JAK3 or anti-STAT5 antibody.

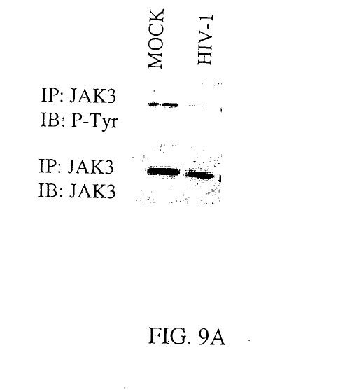

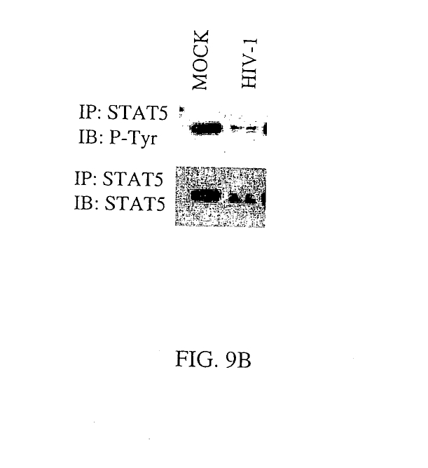

FIG. 9A shows that in HIV-infected cells as compared to Mock-infected cells, activation of JAK3, as shown by tyrosine phosphorylation of JAK3, was inhibited. In addition, FIG. 9A indicates an inhibition of JAK3 expression levels in HIV infected T cells as compared to Mock-infected T cells. FIG. 9B shows that in HIV-infected cells as compared to Mock infected cells, JAK3 kinase activity, as indicated by phosphorylation of the substrate STAT5 by JAK3, was inhibited.

### EXAMPLE 9

The following experiment shows that JAK3 kinase activity is completely inhibited in anti-CD3 stimulated T cells isolated from HIV-infected patients.

In this experiment, peripheral blood T cells were isolated from the venous blood of an HIV-positive patient (child) donor who had a CD4⁺ T cell count of greater than 500 cells/mm³ (See Example 6). Peripheral blood T cells isolated 5 from a healthy, non-infected donor served as a control. 0.5×10⁶ cells per-well (24 well culture plate) were incubated at 37° C. in an anti-CD3 mAb coated (anti-CD3, OKT3, 10 μg/ml) plate for in the presence and absence of rIL-2 (20 U/ml). Cells were harvested at the designated times and Western blots using anti-JAK3, anti-actin, or anti phosphoSTAT5 (pSTAT5) were prepared as described in Example 3.

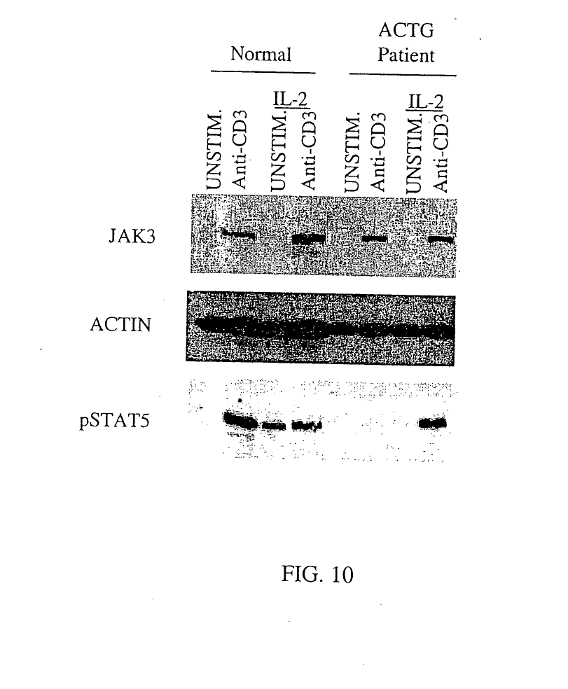

FIG. 10 shows that in this HIV-infected patient, although the level of JAK3 is not significantly inhibited in the T cells after stimulation with anti-CD3, the JAK3 kinase activity, as indicated by the phosphorylation of the STAT5 substrate, was completely inhibited after anti-CD3 stimulation. In contrast, JAK3 kinase activity in T cells in the normal control patient was intact after stimulation with anti-CD3. FIG. 10 also shows that addition of IL-2 to the culture restored the JAK3 kinase activity to the T lymphocytes of the HIV-infected patients.

### EXAMPLE 10

The following example demonstrates that ligation of CD4 prior to T cell receptor-mediated activation of a T cell inhibits JAK3 kinase activity, and that such inhibition is reduced by contacting the T cells with IL-2.

In this experiment, CD4⁺ T cells were isolated and stimulated through TCR/CD3 with or without prior CD4 ligation as described in Example 1, and JAK3 kinase activity, indicated by phosphorylation of STATS, was determined.

Briefly, purified CD4⁺ T lymphocytes were stimulated through TCR/CD3 with or without prior CD4 ligation as described in Example 1.20 U/ml IL-2 was added to the half of the cultures, and JAK3 kinase activity was determined as described in Examples 8 and 9.

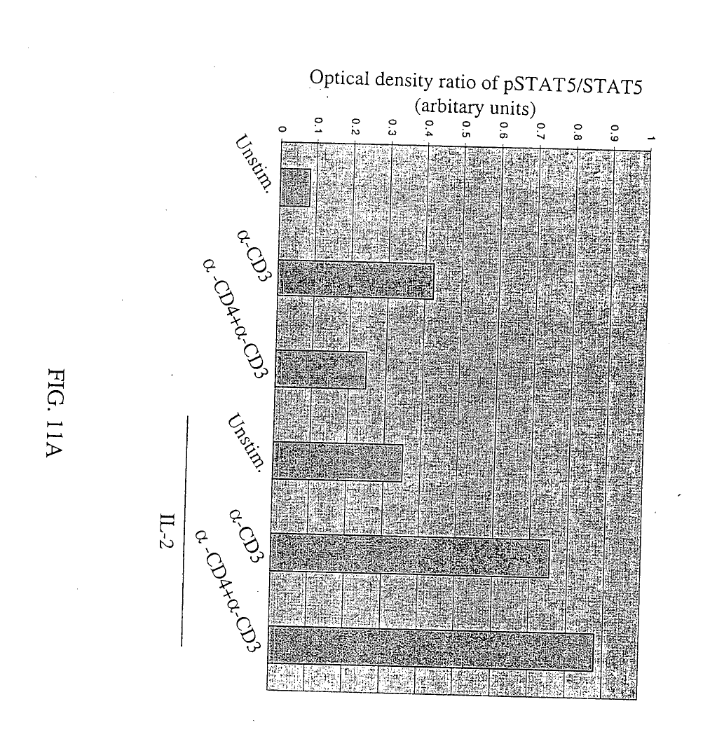

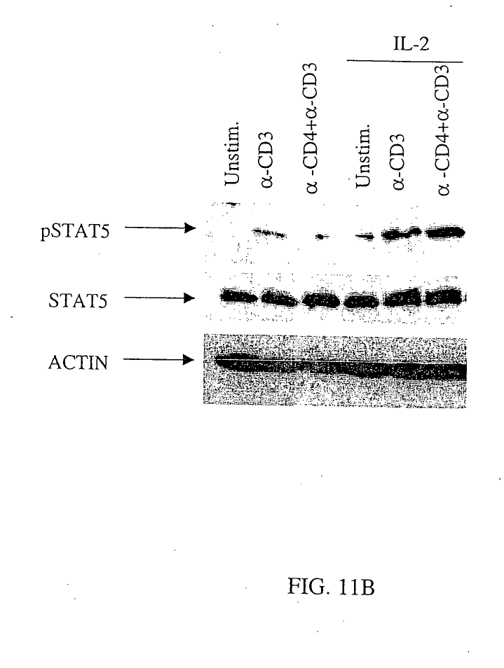

FIG. 11A shows the results of this experiment, presented as the O.D. ratio of pSTAT5 to STAT5 levels. The immunoblot for this experiment is shown in FIG. 11B. FIGS. 11A and 11B show that in CD4⁺ T lymphocytes in which CD4 was ligated prior to stimulation by anti-CD3, JAK3 kinase activity, as indicated by phosphorylation of STAT5, is significantly inhibited. Addition of IL-2 to the cultures restores/increases the JAK3 kinase activity in these cells.

### EXAMPLE 11

### Inhibition of STAT5 Transactivation

WE17/10 T cells (obtained from AIDS reagent program) were electroporated with a STAT5-responsive luciferase vector and an expression vector encoding for HIV-1 Nef, Vpr, or Vpu or a control expression vector. Luciferase activity was monitored with a kit purchased from Promega. As seen in FIG. 12A, the presence of Nef significantly inhibited STAT5 transactivation. Vpr and Vpu encoding expression vectors, however, provided for no significant inhibition of STAT5 transactivation in comparison to a control expression vector.

Additionally, WE17/10 T cells were mock infected or infected with NL4-3 or NL4-3 deleted in vpr, vpu, or nef. At 5 days post infection, the cells were harvested, lysed, and the postnuclear lystaes were analyzed by SDS-PAGE and Western blot with the appropriate antibodies. The optical density (O.D.) of each band was determined and the ratio of JAK3/actin (FIG. 12B), STAT5/actin (FIG. 12C), and pSTAT5/actin (FIG. 12D) was plotted. Notably, the inhibition of STAT5 expression and phosphorylation was the least with NL4-3 that lacked nef.

HeLa cells were also transfected with vectors containing an HIV LTR driven luciferase, IL-2 receptor chains and JAK3. The cells were also transfected with a vector with or without Nef and cultured in the presence or absence of IL-2. As depicted in FIG. 12E, little LTR activity (luciferase) is noted in the absence of IL-2, but the addition of IL-2 leads to a significant increase in activity. The presence of Nef, however, reduces this activity to near the activity levels without IL-2. Therefore, Nef alone is sufficient to alter LTR activity.

### EXAMPLE 12

### STAT5 Binding Sites in HIV-1 Long Terminal Repeats (LTRS)

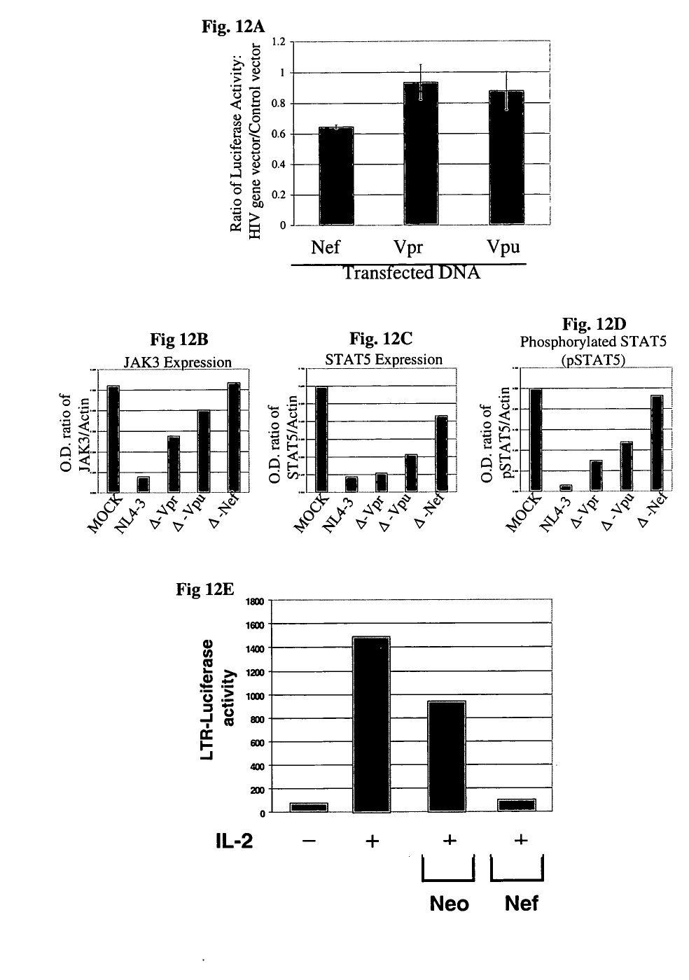

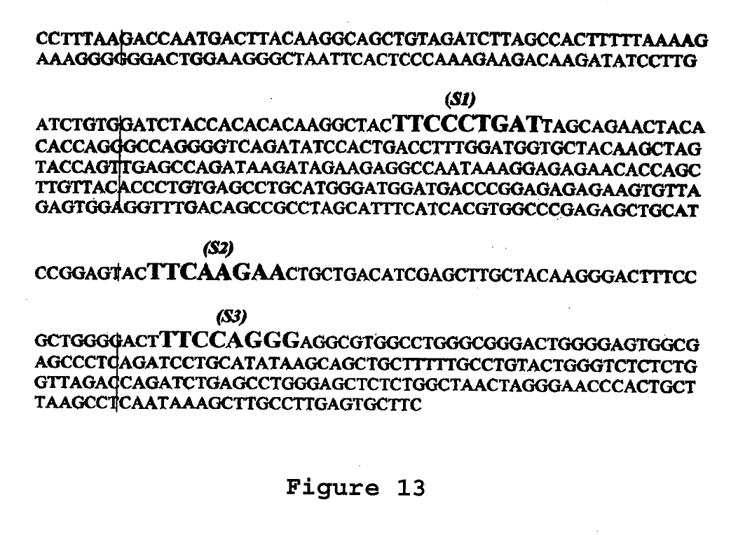

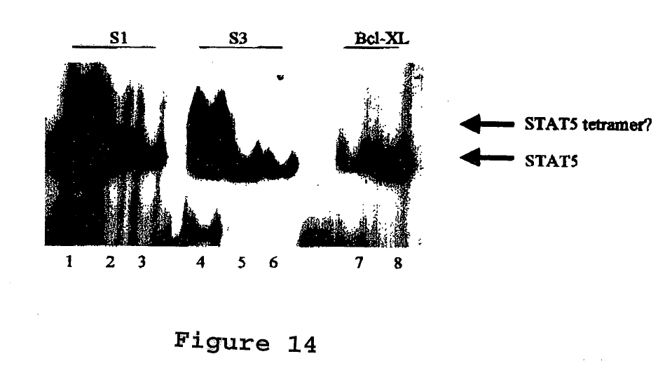

Examination of the 3′ LTR of HIV-1 reveals the presence of three potential STAT5 binding sites, which approximate the consensus STAT5 binding site (TTCNNNGAA, SEQ ID NO: 1; see FIG. 13). To determine if STAT5 protein binds to these potential STAT5 binding sites, electromobility shift assays (EMSAs) were performed. Double stranded oligonucleotides corresponding to the proposed STAT-binding sites S1 (5′-CACAAGGCTACTTCCCTGATTGGCAGAACTA-3′; SEQ ID NO: 2) and S3 (5′-CGCTGGGGACTTTCCAGGGAGGCGTGGCCTG-3′; SEQ ID NO: 3) and the putative STAT5 binding site within the human Bcl-XL gene promoter (5′-GACTTTCCGAGGAAGGCATTTCGGAGAAGAC-3′; SEQ ID NO: 4; Kirito, K., et al. (2002) J. Biol. Chem., 277:8329-8337) were generated by contacting the above oligonucleotides with the corresponding complementary strand, heating to 95° C., and slowly cooling to ambient temperature. The oligonucleotides were labeled with ³²P by incubating with γ-³²P-ATP (3000 Ci/mmol) and T4 polynucleotide kinase (New England Biolabs; Beverly, Mass.). The oligonucleotides (approximately 50,000-100,000 counts per minute; cpm) were contacted with 5-10 μg of WE17/10 or CD4+ primary T cells nuclear extract. The incubation between oligonucleotides and nuclear extract was performed in a binding buffer (10 mM Tris-Cl, pH 7.4; 50 mM NaCl; 4% glycerol; 0.5 mM dithiothreitol; 1 mM MgCl₂; 0.5 mM EDTA, and 1 μg poly dI-dC) at room temperature for 20 minutes. Optionally, 50-fold or 100-fold excess of unlabeled oligonucleotide was incubated with the nuclear extract for 10 minutes prior to the addition of labeled oligonucleotides. Samples were then mixed with loading buffer (25 mM Tris, pH 7.5; 4% glycerol) and electrophoresed on native 6% polyacrylamide gels at 150 V for 3 hours in 0.5× Tris-borate-EDTA. Gels were dried and exposed to Super RX film (Fuji; Japan) at −70° C. with intensifying screens. As seen in FIG. 14, both S1 and S3 demonstrated affinity for STAT5 which could be specifically competed away with excess unlabeled oligonucleotide. The higher complexes most prominently seen in lanes 1 and 4 of FIG. 14 are likely reflect the formation of STAT5 tetramers.

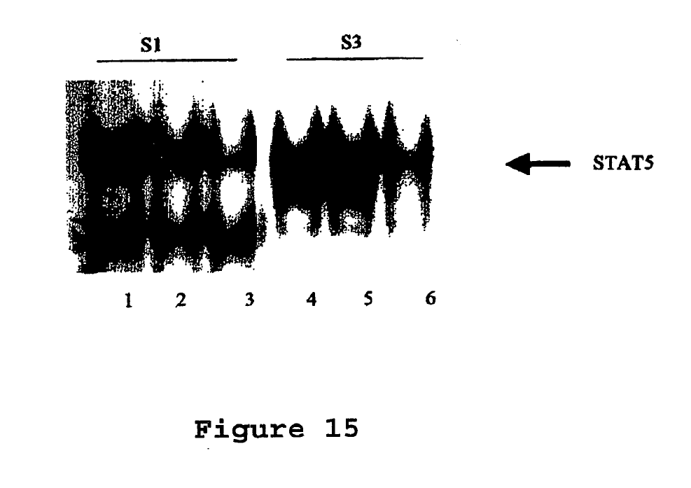

To further confirm that the protein binding the oligonucleotides in FIG. 14 is indeed STAT5, the EMSA was performed, as described above, with antibodies specific for STAT5. Specifically, 5 μg of WE17/10 nuclear extract was incubated with 0, 0.5 or 1.0 μg of anti-STAT5 antibody for 30 minutes at room temperature. Following the incubation, the complexes were incubated with ³²P labeled oligonucleotides representing S1 and S3 for 15 minutes at room temperature and then analyzed by EMSA. The presence of the anti-STAT5 antibody decreased the amount of STAT5 bound to the oligonucleotides thereby indicating that the binding of the STAT5 antibody inhibited the binding of STAT5 to the oligonucleotide (see FIG. 15).

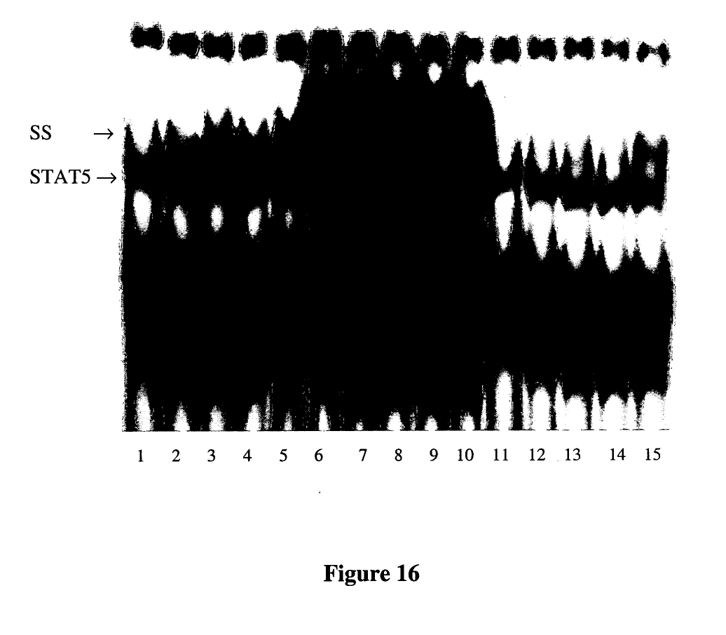

Additionally, an EMSA was performed wherein ³²P labeled (50,000 cpm/reaction) oligonucleotides corresponding to S1, S2 (5′-ATCCGGAGTACTTCAAGAACTGCTGACATC-3′; SEQ ID NO: 6), and the putative STAT5 binding site within the Bcl-XL gene promoter were first incubated with 5 μg of nuclear extract from CD4+, PHA-activated, IL-2 stimulated T cells for 30 minutes at room temperature. Notably, the consensus STAT5 binding site is not exclusive to STAT5 and may be bound by other proteins. Thus, after the incubation period, antibodies specific for STAT1 (33-1400 Zymed Laboratories, Inc; San Francisco, Calif.), STAT3 (06-596 Upstate Biotechnology; Lake Placid, N.Y.), the carboxy terminus of STAT5 (06-588 Upstate Biotechnology), or the SH2 and SH3 (internal) domains of STAT5 (S21520 BD Biosciences/Transduction Laboratories; Franklin Lakes, N.J.) were added to the complexes, allowed to bind for 20 minutes at room temperature, and then analyzed by EMSA. Notably, a supershift indicating antibody binding is seen prominently in lanes 5 and 15 of FIG. 16 indicating the presence of STAT5 in the complex with the S2 and Bcl-XL oligonucleotides. The data from the experiments with S1 are inconclusive as any supershift is masked by the oligonucleotide and nuclear extract alone (see lane 6).

### EXAMPLE 13

### STAT5 Induction of LTR-Luciferase Activity

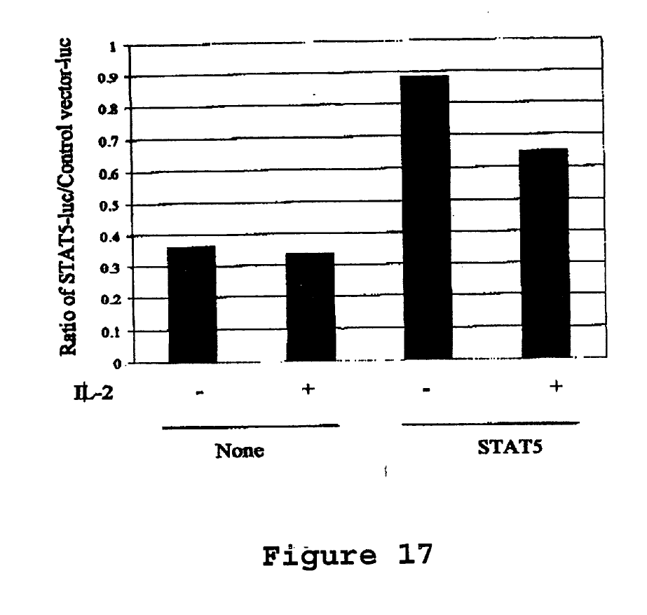

Resting CD4⁺ T cells were transfected with a construct containing luciferase under the control of HIV-1 LTR or a control luciferase construct (Dual-luciferase© Reporter Assay System, Promega, Madison, Wis.). The cells were also optionally transfected with an expression vector encoding STAT5. After incubation at 37° C. for 6 hours, IL-2, which is known to activate STAT5, was optionally added for 1 hour and then cells were lysed. The luciferase activity of the cellular lysates was subsequently measured. As seen in FIG. 17, the over-expression of STAT5 increased activity from the HIV-1 LTR as noted by the increase in luciferase activity. Moreover, the addition of IL-2 did not further increase the activity from the HIV-LTR and, in fact, decreased the activity from the LTR when present in combination with STAT5. High levels of STAT5 plus IL-2 have been, however, determined to result in an increase in cell death in resting T cells, thus, providing a potential explanation for the decrease in the activity when both agents were used in combination.

### EXAMPLE 14

### Chromatin Immunoprecipitation (ChIP) Assay

The ChIP assay was performed as previously described (He, G. and Margolis, D. M. (2002) Mol. Cell. Biol., 22:2965-2973) with some modifications. Briefly, activated, HIV-1-infected, IL-2 stimulated CD4⁺ T cells or TNF-α stimulated J1.1 cells (TNF-α inducible, latently infected Jurkat T cells) were fixed in 1% formaldehyde for 10 minutes at 37° C. After crosslinking, the cells were washed twice with ice-cold D-PBS containing a protease inhibitor cocktail (Sigma, St. Louis, Mo.). The cells were then lysed in 200 μL of SDS Lysis Buffer (Upstate Biotechnology) containing 5 μL of protease inhibitor cocktail (Sigma) per 5×10⁶ cells. Lysates were diluted with 2 ml of CHIP dilution buffer (Upstate Biotechnology) in a 30-ml vial and subjected to sonication for four 15-second pulses with 1 minute pauses utilizing an intermediate tip ultrasonicator. The sonicated lysates were transferred to two 1.5-ml microcentrifuge tubes per sample. Soluble chromatin was collected as the supernatant after a 10-minute centrifugation at 13,000 rpm and 4° C. Chromatin fragmentation was confirmed by agarose gel electrophoresis. Lysates were incubated with 50 μl of each salmon sperm DNA-protein A-agarose and salmon sperm DNA-protein G-agarose beads (Upstate Biotechnology) for 1 hour at 4° C. Next, 10% of the total lysate was set aside to verify the presence of chromatin prior to immunoprecipitation. The remaining lysate was divided equally between and incubated on a rotating platform with 5 μl of anti-STAT1 (33-1400 Zymed Laboratories, Inc), anti-STAT3 (Zymed Laboratories, Inc), anti-STAT5 (BD Biosciences/Transduction Laboratories), anti-NFκB (Santa Cruz Biotechnology; Santa Cruz, Calif.), or rabbit preimmune immunoglobulin G (IgG) serum (Sigma), as appropriate, for two hours at 4° C. Notably, there are 2 NFκB binding sites in the HIV-1 LTR. In an activated T cell, NFκB binds to these sites, allowing proviral transcription to begin.

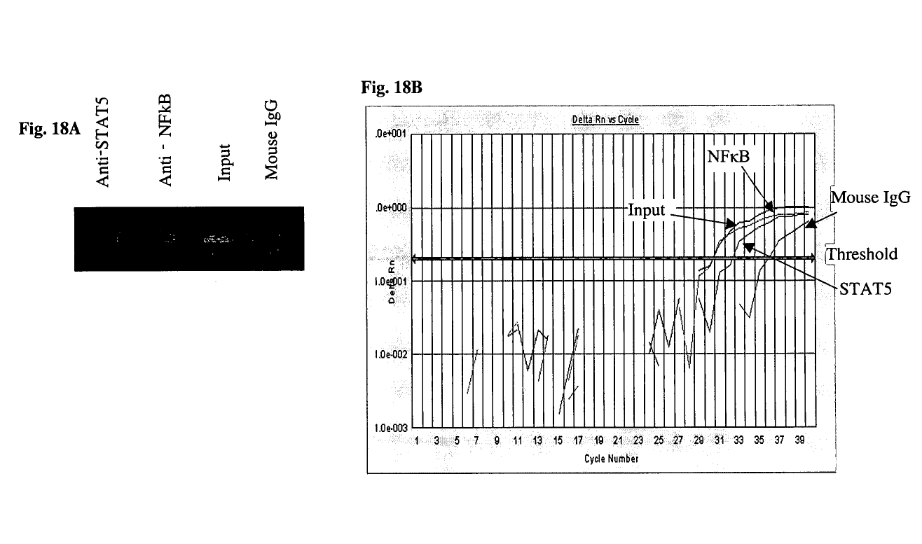

Immunoprecipitates were incubated with 25 μl of each salmon sperm DNA-protein A-agarose and salmon sperm DNA-protein G-agarose beads (Upstate Biotechnology) overnight at 4° C. Agarose beads were recovered by centrifugation and washed sequentially for 5 minutes with 1 ml of each of the following four buffers (Upstate Biotechnology): low-salt wash buffer, high-salt wash buffer, LiCl wash buffer, and Tris-EDTA buffer twice. Immunoprecipitated DNA was eluted with 500 μl of elution buffer (1% sodium dodecyl sulfate [SDS], 0.1 M NaHCO₃). The reversal of DNA-protein cross-linking was performed by incubating the eluates at 65° C. overnight. The soluble chromatin fraction was then incubated with 15 μg of proteinase K (Roche, Germany) at 56° C. for 1 hour. DNA was extracted in phenyl-chloroform-isoamyl alcohol, precipitated in ethanol, washed, and resuspended in 50 μl of TE buffer. The extracted DNA was subsequently amplified by PCR with HIV-1 LTR specific primers that detect both the 2^{nd} and 3^{rd} consensus sites (S2 and S3). Additionally, a fraction of cellular lysate that was not subjected to immunoprecipitation was subjected to PCR (“Input”). The PCR products are seen in FIG. 18. NFκB (a transcription factor known to bind to the HIV-1 LTR) was used as a positive control and demonstrates the feasibility of this assay to detect transcription factor binding to the LTR in vivo. It is worth noting that this is the first time, as far as we are aware, that binding of any transcription factor to the HIV-1 LTR has been demonstrated in vivo. Rabbit IgG is an isotype control antibody and, as expected, showed minimal binding. Notably, STAT5 demonstrated significant binding to the LTR of HIV-1 similar to that seen for NFκB. Real time quantitative PCR was also conducted using DNA that bound to STAT5 or NFκB or to proteins immunoprecipitated by the isotype control antibody. STAT5 and NFκB curves demonstrated fewer amplification cycles required to reach threshold levels compared to mouse IgG isotype control, demonstrating significant levels of STAT5 and NFκB binding to the LTR in vivo.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for preventing the establishment of a latent infection by a microbe in a host, comprising administering to the host an effective amount of at least one latency prevention agent.

2. The method of claim 1, wherein said latency prevention agent is selected from the group consisting of: pentostam, rapamycin, and derivatives and analogs thereof.

3. The method of claim 1, wherein the composition is administered during an acute infection.

4. The method of claim 1, wherein the composition is administered during a structured treatment interruption.

5. The method of claim 1, wherein the microbe is selected from the group consisting of: herpes viruses, lentiviruses, human immunodeficiency virus-1 (HIV-1), HIV-2, feline immunodeficiency virus (FIV), simian immunodeficiency virus (SIV), hepatitis B virus (HBV), Epstein-Barr virus (EBV), Mycobacterium tuberculosis (TB), Schistosoma flatworms, Schistosoma mansoni, S. intercalatum, S. haematobium, S. japonicum and S. mekongi, and Toxoplasma gondii.

6. The method of claim 5, wherein the microbe is HIV-1.

7. The method of claim 2, wherein the composition comprises pentostam and rapamycin.

8. The method of claim 1, wherein the number of cells latently infected by said microbe is reduced.

9. The method of claim 1, further comprising the administration of anti-microbial agents.

10. The method of claim 9, wherein said anti-microbial agents are anti-viral agents.


SEQUENCE LISTING (11 sequences)

SEQ ID NO: 1 - DNA, 9 bp, Artificial Sequence
  Consensus STAT5 binding site
ttcnnngaa

SEQ ID NO: 2 - DNA, 31 bp, Artificial Sequence
  Synthetic Sequence
cacaaggctacttccctgattggcagaacta

SEQ ID NO: 3 - DNA, 31 bp, Artificial Sequence
  Synthetic Sequence
cgctggggactttccagggaggcgtggcctg

SEQ ID NO: 4 - DNA, 31 bp, Artificial Sequence
  Synthetic Sequence
gactttccgaggaaggcatttcggagaagac

SEQ ID NO: 5 - DNA, 616 bp, Human Immunodeficiency Virus
cctttaagaccaatgacttacaaggcagctgtagatcttagccactttttaaaagaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 6 - DNA, 30 bp, Artificial Sequence
  Synthetic Sequence
atccggagtacttcaagaactgctgacatc

SEQ ID NO: 7 - DNA, 563 bp, Human T-Lymphotropic Virus-1
cagggcccagactagggctctgacgtctccccccggagggacagctcagcaccggctcaggctaggccctgacgtgtccccctgaagacaaatcataagctcagacctccgggaagccaccggaaccacccatttcctccccatgtttgtcaagccgccctcaggcgttgacgacaacccctcacctcaaaaaacttttcatggcacgcatatggctgaataaactaacaggagtctataaaagcgtggagacagttcaggagggggctcgcatctctccttcacgcgcccgccgccctacctgaggccgccatccacgccggttgagtcgcgttctgccgcctcccgcctgtggtgcctcctgaactgcgtccgccgtctaggtaagttcagagctcaggtcgagaccgggcctttgtccggcgctcccttggagcctgcctagactcagccggctctccacgctttgcctgaccctgcttgctcaactctgcgtctttgtttcgttttctgttctgcgccgctacagatcgaaagttccacccctttccctttcattcacg

SEQ ID NO: 8 - DNA, 360 bp, Feline Immunodeficieny Virus
tgggatgagtattggaaccctgaagaaatagaaagaatgcttatggactagggactgtttacgaacaaatgataaaaggaaatagctgagcatgactcatagttaaagcgctagcagctgcctaaccgcaaaaccacatcctatggaaagcttgctaatgacgtataagttgttccattgtaagagtatataaccagtgctttgtgaaacttcgaggagtctctttgttgaggacttttgagttctcccttgaggctcccacagatacaataaatatttgagattgaaccctgtcgagtatctgtgtaatcttttttacctgtgaggtctcggaatccgggccgagaacttcgcagttgg

SEQ ID NO: 9 - DNA, 690 bp, Simian Immunodeficiency Virus
tggatgggatatattactctgaaagaagagaaaagatcctgaatttgtatgccttgaacgagtggggaataatagatgattggcaagcttactcaccaggcccggggataaggtacccgagagtctttggcttctgctttaagctagtcccagtggacctgcatgaggaggcacgcaactgtgagagacactgtctgatgcatccagcacagatgggggaagatcctgatggaatagatcatggagaagtcttggtctggaagtttgacccgaagttggcggtggagtaccgcccggacatgtttaaggacatgcacgaacatgcaaagcgctagtgtcagcactttgcggttgggactttccgccagggactttccacagtgggtggatcggaggcggtacaggggcggtactgggagtggctttcccctcagagctgcataaaagcagatgctcgctggcttgtaactcagtctcttactaggagaccagctagagcctgggtgttcgctggttagcctaacccggttggccaccgggggtaaggactccttggcttcatatagctcaataaacctgctcgcttagtcgctatattggagtcaagtgctcattgctgcgccgagcctctagaggtgaacctctcttactgggttctcctgtacccaggtgggagaaactccagcagtg

SEQ ID NO: 10 - protein, 794 aa, Homo Sapiens
MAGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 11 - protein, 787 aa, Homo Sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS